(19) United States
(12) Patent Application Publication
(10) Pub. No.: US 2022/0154134 A1
(43) Pub. Date: May 19, 2022

(54) PROBIOTIC BIOFILM COMPOSITIONS AND METHODS OF PREPARING SAME

(71) Applicant: MYBIOTICS PHARMA LTD., Rehovol (IL)

(72) Inventors: David DABOUSH, Mishmar David (IL); Dorit ROZNER, Gedera (IL); Stephanie COHEN, Ashdod (IL); Hila ELIYAHU, Jerusalem (IL)

(21) Appl. No.: 17/598,878

(22) PCT Filed: Mar. 29, 2020
(86) PCT No.: PCT/IL2020/050380

§ 371 Date: Sep. 28, 2021

Related U.S. Application Data
(63) Continuation-in-part of application No. 16/368,030, filed on Mar. 28, 2019, now Pat. No. 10,709,744.
(60) Provisional application No. 62/827,931, filed on Apr. 2, 2019.

(51) Int. Cl. C12N 1/20; A61K 35/745; A61K 35/747; A61P 1/00

(52) U.S. Cl. CPC C12N 1/205; A61K 35/745; C12R 2001/225; A61P 1/00; A61K 35/747

(57) ABSTRACT

The present invention is directed to compositions including probiotic bacteria in the form of biofilm, wherein the biofilm includes at least two bacterial species. Further provided are a method of using the composition of the invention, and a method of making same.

### CROSS-REFERENCE TO RELATED APPLICATIONS

This application claims the benefit of priority of U.S. patent application Ser. No. 16/368,030 titled “PROBIOTIC BIOFILM SUPPOSITORIES”, filed Mar. 28, 2019, and U.S. Provisional Patent Application No. 62/827,931 titled “BIOFILM COMPOSITIONS AND METHODS OF PREPARING SAME”, filed Apr. 2, 2019, the contents of which are incorporated herein by reference in their entirety.

### FIELD OF INVENTION

The present invention is directed to the field of probiotics delivery.

### BACKGROUND

A healthy microbiota requires bacterial colonization which provides the host multiple benefits including resistance to a broad spectrum of pathogens, essential nutrient biosynthesis and absorption, and immune stimulation that maintains a healthy gut epithelium and an appropriately controlled systemic immunity. In settings of dysbiosis or disrupted symbiosis, microbiota functions can be lost or deranged, resulting in increased susceptibility to pathogens, altered metabolic profiles, or induction of proinflammatory signals that can result in local or systemic inflammation or autoimmunity.

Urogenital infections such as yeast vaginitis, bacterial vaginosis, and urinary tract infection remain a major medical problem in terms of the number of women afflicted each year. These diseases affect the organs and tissues related to the reproductive system.

For all women up to the age of 40, microbiota is mainly represented by lactobacilli, and in pathological complications of the urogenital tract of women, the microbial composition of the biocoenosis is characterized by a decrease in the number of lactobacilli and their replacement by pathogenic anaerobic microorganisms. A change in the vaginal flora characterized by the decrease of lactobacilli appears to be the major factor causing the syndrome bacterial vaginosis.

Although antimicrobial therapy is generally effective at eradicating these infections, there is still a high incidence of recurrence. The patient's quality of life is affected, and many women become frustrated by the cycle of repeated antimicrobial agents whose effectiveness is diminishing due to increasing development of microbial resistance.

Regular administration of a Lactobacillus strain with ability to colonize vaginal tissue can be an alternative solution for this problem. It has been shown that promising results can be obtained by using a treatment of both antibiotics and probiotics in parallel.

The most common Lactobacillus sp. in the lower genital tract are Lactobacillus iners, Lactobacillus crispatus, Lactobacillus jensenii and Lactobacillus gasseri. In recent years, several studies have pointed out on the vital role lactobacilli have in maintaining vaginal health, while preventing genital tract infections. The beneficial effect of Lactobacillus crispatus on colitis has also been reported.

However, it is well established in numerous studies that commercial probiotics both supplements of planktonic powders and fermented foods exert little to no health effect and lack the ability to directly deliver viable bacteria to the rectal area or the vaginal area.

There is a need for a vaginal suppositories formulation in which the probiotics are viable under the vaginal conditions, are able to adhere to the vaginal epithelial cells for a successful colonization. Moreover, it is important that such formulations are resistant to the common antibiotics used in the treatment.

### SUMMARY

According to a first aspect, there is provided a composition comprising a first lipophilic carrier, a co-cultured probiotic bacteria in the form of a dried biofilm comprising Lactobacillus crispatus and at least one additional bacterial species selected from the group consisting of: L. gasseri, L. jensenii, and L. rhamnosus, and a first agent comprising: an antibiotic agent, a pH adjusting agent, or both.

According to another aspect, there is provided a composition comprising: a co-cultured probiotic bacteria in the form of a dried biofilm comprising: (i) Faecalibacterium prausnitzii and at least one additional bacterial species selected from the group consisting of: Blautia obeum, Bl. coccoides, Bacteroides vulgatus, and Dorea (Eubacterium) formicigenerans; (ii) Lactobacillus crispatus and at least one additional bacterial species selected from the group consisting of: L. gasseri, L. jensenii, and L. rhamnosus; or (iii) Bifidobacterium adolescentis and at least one additional bacterial species selected from the group consisting of: Bif. Longum sub.longum, and Bif. breve;

According to another aspect, there is provided a method for modulating the flora in a subject in need thereof, comprising administering to the subject a therapeutically effective amount of the herein disclosed composition, thereby modulating the flora in the subject.

According to another aspect, there is provided a method for preparing the herein disclosed composition, the method comprises the steps of: (a) inoculating a growth medium comprising a particle with L. crispatus; (b) incubating the particle with L. crispatus from step (a) under conditions suitable for allowing L. crispatus to attach to the particle; (c) inoculating the particle of step (b) with at least one additional bacterial species; and (d) culturing the inoculated particle of step (c) under conditions suitable for forming a biofilm comprising L. crispatus and at least one additional bacterial species.

According to another aspect, there is provided a method for preparing the herein disclosed composition comprising the steps of:
inoculating a growth medium comprising a particle with a first bacteria selected from: (i) L. crispatus; (ii) Bif. adolescentis; or (iii) F. prausnitzii;
incubating the particle with the first bacteria under conditions suitable for allowing the first bacteria to attach to the particle;
inoculating the particle with at least one additional bacterial species; and
culturing the inoculated particle under conditions suitable for forming a biofilm comprising: (i) L. crispatus; (ii) Bif. adolescentis; or (iii) F. prausnitzii, and at least one additional bacterial species.

In some embodiments, the co-cultured probiotic bacteria in the form of a dried biofilm and the first agent comprising an antibiotic agent are homogeneously dispersed within the composition.

In some embodiments, the co-cultured probiotic bacteria in the form of dried biofilm is 10% to 50% (w/w) of the total composition.

In some embodiments, the co-cultured probiotic bacteria in the form of dried biofilm is attached to a particle.

In some embodiments, the particle is selected from the group consisting of: seeds, MCC, dicalcium phosphate, a polysaccharide, and any combination thereof.

In some embodiments, the composition further comprises a second layer.

In some embodiments, the second layer comprises a second lipophilic carrier, a second agent or both.

In some embodiments, the release of the co-cultured probiotic bacteria in the form of dried biofilm is slower than the release of the second agent.

In some embodiments, any one of the first agent and the second agent is an antibiotic.

In some embodiments, the composition is for use in the treatment of bacterial vaginosis.

In some embodiments, the composition further comprises a stabilizer, a preservative, a lubricant, a viscosity modifying agent, a buffering agent, fatty acids, and combinations thereof.

In some embodiments, the composition is formulated for a delivery route selected from the group consisting of: oral, vaginal, rectal, and topical.

In some embodiments, the composition is in the form of a suppository.

In some embodiments, the flora is a vaginal flora, a gut flora, or a skin flora.

In some embodiments, the method is for preventing or treating a dysbiosis related condition or an intestinal or metabolic disease in a subject in need thereof.

In some embodiments, the dysbiosis related condition or an intestinal and metabolic disease is selected from the group consisting of: bacterial vaginosis, a urogenital infection, ulcerative colitis, inflammatory bowel disease (IBD), Crohn's disease, colorectal cancer, obesity, and celiac disease.

In some embodiments, the subject is afflicted with or at risk of developing bacterial vaginosis, a urogenital infection, dysbiosis, ulcerative colitis, an inflammatory bowel disease (IBD), Crohn's disease, or any combination thereof.

In some embodiments, when the first bacteria is L. crispatus the at least one additional bacterial species is selected from the group consisting of: L. jensenii, L. gasseri, and L. rhamnosus.

In some embodiments, when the first bacteria is Bif. adolescentis the at least one additional bacterial species is selected from the group consisting of: Bif. Longum sub.longum, and Bif. breve.

In some embodiments, when the first bacteria is F. prausnitzii the at least one additional bacterial species is selected from the group consisting of: Bl. obeum, Bl. coccoides. Bac. vulgatus, and Dorea (Eubacterium) formicigenerans.

In some embodiments, the first bacteria is F. prausnitzii and the inoculating steps are performed simultaneously.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIGS. 1A-1G present pictures of the different suppository formulations presented in table 1 (FIGS. 1A-1F), and a diagram of the experimental design and assays that were performed to optimize growth of bacteria in biofilm using bacteria in form of biofilm as well as to evaluate bacteria in form of biofilm developmental phase. The use of pH 3.5 in the pH resistance assay was determined to have a pH value close to the pH that prevails in woman vagina (pH 4-5). Susceptibility of planktonic bacteria to the resistance assays was also determined and results were subsequently compared to bacteria in form of biofilm results. CFU, colonies forming units (FIG. 1G).

FIG. 2 presents a bar graph of acid resistance of planktonic bacteria of L. iners.

FIG. 3 presents a bar graph of acid resistance of L. iners Bacteria in form of biofilm following growth in a small-scale set-up. Aerobic and anaerobic conditions were examined as well as aeration condition (static vs. stir).

FIG. 4 presents a bar graph of acid resistance of L. iners Bacteria in form of biofilm following growth in a medium scale set-up.

FIG. 5 presents a bar graph of antibiotics resistance of L. iners Bacteria in form of biofilm following growth in a medium-scale set-up. ‘Control’ refers to Bacteria in form of biofilm that was not exposed to antibiotics. Numbers in the x-axis are antibiotics concertation in μg/mL.

FIG. 6 presents a bar graph of acid resistance of planktonic bacteria of L. jensenii.

FIG. 7 presents a bar graph of acid resistance of L. jensenii Bacteria in form of biofilm following growth in a small-scale set-up while examining two agitation speeds (70 and 130 rpm).

FIG. 8 presents a bar graph of acid resistance of L. jensenii Bacteria in form of biofilm following growth in a medium-scale set-up while examining two agitation speeds (70 and 130 rpm).

FIG. 9 presents a bar graph of antibiotics resistance of L. jensenii Bacteria in form of biofilm following growth in a medium-scale set-up. ‘Ctrl’ refers to Bacteria in form of biofilm that was not exposed to antibiotics. Numbers in the x-axis are antibiotics concertation in μg/mL.

FIG. 10 presents a bar graph of acid resistance of planktonic bacteria of L. crispatus.

FIG. 11 presents a bar graph of acid resistance of L. crispatus Bacteria in form of biofilm following growth in a small-scale set-up while examining agitation and non-agitation conditions.

FIG. 12 presents a bar graph of acid resistance of L. crispatus Bacteria in form of biofilm following growth in a medium scale set-up.

FIG. 13 presents a bar graph of antibiotics resistance of L. crispatus Bacteria in form of biofilm following growth in a medium-scale set-up. ‘Ctrl’ refers to Bacteria in form of biofilm that was not exposed to antibiotics. Numbers in the x-axis are antibiotics concertation in μg/mL.

FIG. 14 presents a bar graph of acid resistance of planktonic bacteria of L. gasseri.

FIG. 15 presents a bar graph of acid resistance of L. gasseri Bacteria in form of biofilm following growth in a small-scale set-up while examining two agitation speeds (70 and 130 rpm).

FIG. 16 presents a bar graph of acid resistance of L. gasseri Bacteria in form of biofilm following growth in a medium scale set-up.

FIG. 17 presents a bar graph of antibiotics resistance of L. iners Bacteria in form of biofilm following growth in a medium-scale set-up. ‘Ctrl’ refers to Bacteria in form of biofilm that was not exposed to antibiotics. Numbers in the x-axis are antibiotics concertation in μg/mL.

FIG. 18 presents a bar graph of acid resistance of planktonic bacteria of L. rhamnosus.

FIG. 19 presents a bar graph of acid resistance of L. rhamnosus Bacteria in form of biofilm following growth in a small-scale set-up while examining two agitation speeds (70 and 130 rpm).

FIG. 20 presents a bar graph of acid resistance of L. rhamnosus Bacteria in form of biofilm following growth in a medium scale set-up.

FIG. 21 presents a bar graph of antibiotics resistance of L. rhamnosus Bacteria in form of biofilm following growth in a medium-scale set-up. ‘Ctrl’ refers to Bacteria in form of biofilm that was not exposed to antibiotics. Numbers in the x-axis are antibiotics concertation in μg/mL.

FIG. 22 presents a bar graph of the effect of cranberries on Bacteria in form of biofilm survival in suppositories for two months. ‘cran’ refers to cranberries; ‘supp’ refers to suppositories.

FIG. 23 presents a bar graph of survival of wet- and dry-Bacteria in form of biofilm after 1 and 3 months in suppositories.

FIG. 24 presents a bar graph of survival of L. plantarum Bacteria in form of biofilm in suppositories containing different ratio of Bacteria in form of biofilm:excipients, 1:5 or 1:10, respectively. Excipients comprised of two oil-based carriers, vegetable butter and cocoa butter. ‘supp’ refers to suppositories.

FIG. 25 presents a bar graph of the effect of different particles on the growth of L. gasseri Bacteria in form of biofilm.

FIG. 26 presents a bar graph comparing the stability of a suppository formulation comprising a combination of Pentasa with Bacteria in form of biofilm. ‘supp A’ refers to L. gasseri and L. rhamnosus biofilm. ‘supp B’ refers to L. gasseri and L. rhamnosus biofilm with Pentasa.

FIG. 27 presents a picture of colonies morphologies of L. rhamnosus (LRh), L. jensenii (LJ) and L. gasseri (LG) as obtained from the co-culture experiment. Bacteria cells were released from the biofilm by vortex before plating on MRS agar plate for CFU counting.

FIGS. 28A-28B present bar graphs of co-culture of bacterial strains. Bacterial population growth and biofilm development (pH resistance) was compared between the strain growth alone and together. Dashed line refers to the threshold for bacterial resistance; Bacterial population that survived above this point is considered resistant to the tested treatment. (28A) present a bar graph of co-culture of strain L. jensenii (LJ) with L. rhamnosus (LRh) (28B) present a bar graph of co-culture of strain L. gasseri (LG) with L. rhamnosus (LRh).

FIGS. 29 presents a picture of colonies morphologies of L. rhamnosus (LRh), L. jensenii (LJ), L. gasseri (LG) and L. crispatus (LCr) as obtained from the co-culture experiment. Bacteria cells were released from the biofilm by vortex before plating on MRS agar plate for CFU counting. Images of the bacteria colonies were taken from the agar plate.

FIGS. 30A-30B present bar graphs of co-culture of three or four bacterial strains. Bacterial population growth and biofilm development (pH resistance) of each strain in the co-culture (the current experiment) was compared to their growth alone (former experiments). Dashed line refers to the threshold for bacterial resistance; Bacterial population that survived above this point is considered resistant to the tested treatment. (30A) presents a bar graph of Co-culture of L. rhamnosus (LRh), L. jensenii (LJ) and L. gasseri (LG) (30B) present a bar graph of Co-culture of L. rhamnosus (LRh), L. jensenii (LJ), L. gasseri (LG), and L. crispatus (LC).

FIG. 31 presents a bar graph showing affinity of the L. gasseri (LG) Bacterial population to various particle size.

FIGS. 32A-32C present bar graphs of the effect of different pH levels and animal-based and non-animal-based growth mediums on the growth and developmental state of LCr (32A), LG (32B), and LJ (32C) cultures.

FIGS. 33A-33C present bar graphs of the effect of animal-based medium and nonanimal1 based medium on growth and development (pH and antibiotics resistance) of LG (33A), LRh (33B), and LCr (33C) Bacterial population. Dashed line refers to the threshold for bacterial resistance; bacterial population that survived above this point is considered resistant to the tested treatment.

FIG. 34 presents a vertical bar graph showing that Bifidobacterium adolescentis (BfA) has an improved growth after 72 h when cocultured with Bifidobacterium longum sub.longum (BLL) and B. breve (BfBr) compare to its growth alone.

FIG. 35 presents a vertical bar graph showing that BfA has an improved growth when cocultured with BfBr compare to its growth alone after 24 h and 72 h of growth.

FIG. 36 presents a vertical bar graph showing that BfA has higher relative abundance (RA) when it is added 24 h prior to the addition of BLL, B. animalis (BB-12), and BfBr. BfA was added either in parallel with BLL and BfBr (‘control’) or 24 h before the addition of BLL and BfBr (‘Advantage to BfA’).

FIG. 37A-37B present bar graphs showing improved growth of BfA (˜1 log difference) when it is added 24 h prior to the addition of BLL, BB-12 and BfBr (‘Advantage to BfA’) compare to its addition in parallel to the other Bifidobacterium species (‘Control’). The relative abundance values (in FIG. 36) are normalized to bacteria count (log scale).

FIG. 38A-38B present vertical bar graphs showing improved growth of BfA (˜1 log difference) when it is added 24 h prior to the addition of BLL, BB-12 and BfBr (‘Advantage to BfA’) compare to its addition in parallel to the other Bifidobacterium species (‘Control’). The relative abundance values (in FIG. 36) are normalized to bacteria count (CFU/mL).

FIG. 39 presents a vertical bar graph showing that Faecalibatcerim prausnitzii (FaP) show an improved growth when cocultured with either Blautia obeum (BlO), Bl. coccoides (BlC) or both, compare to its growth alone.

FIG. 40 presents a vertical bar graph showing that FaP show an improved growth when cocultured with either Bacteroides vulgatus (BaV) or both BaV and Dorea (Eubacterium) formicigenerans (DoF) compare to its growth alone.

FIG. 41 presents a vertical bar graph showing that FaP shows the improved growth when coculture with either Bifidobacterium adolescentis (BfA) or Bacteroides thetaiotaomicron (BaT).

FIG. 42 presents a vertical bar graph showing that FaP is cocultured with Bacteroides spp. (right column), a clear advantage is observed for FaP growth in comparison to its growth in coculture with only Clostridiales spp. (left column).

FIG. 43A-43B present vertical bar graphs showing that FaP has a higher relative abundance (RA) in biofilm (43B) compared to the planktonic part (43A) during 72 h of growth.

### DETAILED DESCRIPTION

According to some embodiments, the present invention provides a composition comprising at least one bacteria in the form of biofilm.

In some embodiments, the biofilm is in the form of dried biofilm or the form of a powder biofilm. In some embodiments, the biofilm is biofilm particles.

As used herein, the term “biofilm particles” refers to bacteria (e.g., probiotic bacteria) in the form of biofilm and in a form of particles.

In some embodiments, the composition comprises a plurality of bacteria. In some embodiments, a plurality is any integer greater than 1, e.g., at least 2, at least 3, at least 4, at least 5, at least 6, etc., or any value and range therebetween. Each possibility represents a separate embodiment of the invention.

In some embodiments, at least one bacterium within the composition produces biofilm. In some embodiments, all bacteria within the composition produce biofilm.

In some embodiments, the composition comprises planktonic bacteria. In some embodiments, the planktonic bacteria do not produce biofilm. In some embodiments, the planktonic bacteria produce a low amount of biofilm compared to the biofilm producing bacteria.

In some embodiments, the composition comprises planktonic bacteria and biofilm producing bacteria, wherein the planktonic bacteria are stuck, entrapped, merged, or embedded, under, onto, or within the biofilm produced by the biofilm producing bacteria.

In some embodiments, the herein disclosed probiotic bacteria in the form of biofilm comprises a mixture of planktonic bacteria and bacteria in the form of biofilm.

### Biofilm Compositions

According to some embodiments, there is provided a composition comprising a first lipophilic carrier, a co-cultured probiotic bacteria in the form of a dried biofilm comprising Lactobacillus crispatus and at least one additional bacterial species selected from the group consisting of: L. gasseri, L. jensenii, and L. rhamnosus, and a first agent comprising: an antibiotic agent, a pH adjusting agent, or both.

In some embodiments, co-cultured probiotic bacteria in the form of a dried biofilm and the first agent comprising an antibiotic agent are homogeneously dispersed within the composition.

According to some embodiments, the composition comprises a bacteria in the form of biofilm comprising: Faecalibacterium prausnitzii and at least one additional bacterial species selected from: Blautia obeum, Bl. coccoides, Bacteroides vulgatus, Dorea (Eubacterium) formicigenerans, or any combination thereof.

According to some embodiments, the composition comprises a bacteria in the form of biofilm comprising: Lactobacillus crispatus and at least one additional bacterial species selected from: L. gasseri, L. iners, L. jensenii, L. rhamnosus, or any combination thereof.

According to some embodiments, the composition comprises a bacteria in the form of biofilm comprising: Bifidobacterium adolescentis and at least one additional bacterial species selected from: Bif. Longum sub.longum, Bif breve and a combination thereof.

As used herein, the term “probiotic” refers to a beneficial or required bacterial strain that can also stimulate the growth of other microorganisms, especially those with beneficial properties (such as those of the vaginal flora and gut flora).

In some embodiments, the biofilm comprises at least one bacterial strain derived from vaginal microflora. In some embodiments, the at least one bacterial strain derived from vaginal microflora is a probiotic bacterium.

In some embodiments, the biofilm comprises at least one bacterial strain derived from gut microflora. In some embodiments, the at least one bacterial strain derived from gut microflora is a probiotic bacterium.

In some embodiments, the biofilm comprises at least one bacterial strain derived from the colon. In some embodiments, the biofilm comprises at least one bacterial strain derived from the stomach. In some embodiments, the biofilm comprises at least one bacterial strain derived from the small intestine.

In some embodiments, the at least one probiotic bacteria is selected from the genera Lactobacillus, Bifidobacterium, Saccharomyces, Enterococcus, Streptococcus, Faecalibacterium, Pediococcus, Leuconostoc, Bacillus, Escherichia coli, or any combination thereof.

Non-limiting examples of gut-derived strains include Lactobacillus rhamnosus GG (LGG), Streptococcus thermophiles, Lactobacillus acidophilus, Bifidobacterium lactis, Bifidobacterium breve, Bifidobacterium longum, Bifidobacterium infantis, Enterococcus faecium, Lactobacillus plantarum, Lactobacillus rhamnosus, Propionibacterium freudenreichii, Bifidobacterium breve, Lactobacillus reuteri, Lactobacillus salivarius, Bifidobacterium infantis, Streptococcus thermophiles, and Faecalibacterium prausnitzii.

In some embodiments, the biofilm comprises at least one Lactobacillus bacterial strain. Non-limiting examples of Lactobacillus include Lactobacillus crispatus, Lactobacillus gasseri, Lactobacillus iners, Lactobacillus jensenii, Lactobacillus rhamnosus, Lactobacillus Lactobacillus rhamnosus GG, Lactobacillus acidophilus, Lactobacillus plantarum, Lactobacillus paracasei, Lactobacillus delbrueckii ssp. Bulgaricus.

In some embodiments, the probiotic bacteria can colonize a vaginal tissue. In some embodiments, the probiotic bacteria are more proficient in colonizing vaginal tissue compared to similar bacteria that are provided in a planktonic form. The degree of improvement of colonization may be measured as an increase in the quantity of bacteria in samples from a tissue treated with biofilm particle-based suppositories compared to a control tissue which is treated with planktonic probiotic bacteria-based suppositories, after a predetermined period of time from administration.

In some embodiments, the bacteria provided herein is generated using the methods as disclosed in PCT/IB2016/000933 and PCT/IL2017/050587 incorporated herein by reference, in its entirety.

In one embodiment, one or more bacterium for generating biofilm provided herein is obtained from a healthy mammal. In one embodiment, the bacterium is obtained from an animal donor. In one embodiment, the donor may be screened for their health status and nutrition habits. In one embodiment, the bacterium is derived from a bacterial strain. In some embodiments, the bacterium is derived from stored bacterial strain. In some embodiments, the plurality of bacteria is derived from frozen bacterial strain. In some embodiments, the bacterium is derived from frozen biofilm. In some embodiments, the bacterium is derived from lyophilized bacterial strain.

In one embodiment, the biofilm comprises at least one bacterial strain derived from a stored microbiota sample. In one embodiment, the biofilm comprises at least one bacterial strain derived from a bacterial colony.

According to some embodiments, the CFU of the composition is 10⁴ to 10¹⁵. According to some embodiments, the CFU of the composition is 10⁴ to 10¹⁴. According to some embodiments, the CFU of the composition is 10⁴ to 10¹³. According to some embodiments, the CFU of the composition is 10⁴ to 10¹². According to some embodiments, the CFU of the composition is 10⁵ to 10¹⁵. According to some embodiments, the CFU of the composition is 10⁵ to 10¹⁴. According to some embodiments, the CFU of the composition is 10⁵ to 10¹³. According to some embodiments, the CFU of the composition is 10⁵ to 10¹². According to some embodiments, the CFU of the composition is 10⁶ to 10¹⁵. According to some embodiments, the CFU of the composition is 10⁶ to 10¹⁴. According to some embodiments, the CFU of the composition is 10⁶ to 10¹³. According to some embodiments, the CFU of the composition is 10⁶ to 10¹². According to some embodiments, the CFU of the composition is 10⁷ to 10¹⁵. According to some embodiments, the CFU of the composition is 10⁷ to 10¹⁴. According to some embodiments, the CFU of the composition is 10⁷ to 10¹³. According to some embodiments, the CFU of the composition is 10⁷ to 10¹². According to some embodiments, the CFU of the composition is 10⁸ to 10¹⁵. According to some embodiments, the CFU of the composition is 10⁸ to 10¹⁴. According to some embodiments, the CFU of the composition is 10⁸ to 10¹³. According to some embodiments, the CFU of the composition is 10⁸ to 10¹².

According to some embodiments, the at least one additional bacterial species is present in the composition in an amount of 10⁴ to 10¹⁵ CFU. According to some embodiments, the at least one additional bacterial species is present in the composition in an amount of 10⁸ to 10¹² CFU.

According to one aspect, the ratio of Lactobacillus crispatus and the at least one additional bacterial species is selected from 1:100000-100000:1, 1:10000-10000:1, 1:1000-100:1, 1:100-1000:1, and 1:100-100:1.

According to some embodiments, the biofilm has at least one feature selected from: acid tolerant, antibiotics-resistant, temperature resistance, and any combination thereof.

In some embodiments, the composition further comprises: (i) a first agent comprising an antibiotic agent; (ii) a first lipophilic carrier, (iii) a pH adjusting agent, or any combination thereof.

In some embodiments, the at least one probiotic bacteria in the form of biofilm is homogeneously dispersed within the first lipophilic carrier, the first agent, or any combination thereof, thereby forming a first layer.

### Lipophilic Carriers

According to some embodiments, the present invention provides a composition comprising at least one probiotic bacteria in the form of biofilm and a first lipophilic carrier, wherein the at least one probiotic bacterium is 10% to 50% (w/w) of the total composition.

In some embodiments, the at least one probiotic bacterium is 12% to 50% (w/w), 15% to 50% (w/w), 20% to 50% (w/w), 12% to 48% (w/w), 12% to 15% (w/w), 12% to 42% (w/w), 12% to 40% (w/w), 15% to 48% (w/w), 15% to 40% (w/w), 20% to 50% (w/w), 20% to 48% (w/w), 20% to 45% (w/w), or 20% to 40% (w/w), of the total composition.

In some embodiments, the first lipophilic carrier and the second lipophilic carrier are solid at room temperature and are each independently characterized by melting point of at least 25° C. In some embodiments, the first lipophilic carrier and the second lipophilic carrier are solid at room temperature and characterized by melting point of at least 26° C., at least 27° C., at least 28° C., at least 29° C., at least 30° C., at least 31° C., or at least 32° C., including any value therebetween.

In some embodiments, the first lipophilic carrier and the second lipophilic carrier have each independently a melting point in the range of 25° C. to 60° C. In some embodiments, the first lipophilic carrier and the second lipophilic carrier have each independently a melting point in the range of 27° C. to 60° C., 30° C. to 60° C., 25° C. to 58° C., 25° C. to 55 C., 27° C. to 58° C., or 27° C. to 55° C., including any range therebetween.

In some embodiments, the first lipophilic carrier and the second lipophilic carrier comprise one or more fatty acids with a saturated content of more than 40%. In some embodiments, the first lipophilic carrier and the second lipophilic carrier comprise one or more fatty acids with a saturated content of more than 41%, more than 45%, more than 48%, or more than 50%, including any value therebetween.

In some embodiments, the first lipophilic carrier and the second lipophilic carrier comprise one or more hydrogenated fats.

As used herein the term “hydrogenated fats” refers to fatty acids that have been chemically altered. In general, hydrogenated fats are oils whose chemical structures were changed to become solid fats.

In some embodiments, the second lipophilic carrier has a melting point at least 5° C., at least 6° C., at least 7° C., at least 10° C., at least 12° C., or at least 15° C., higher than the first lipophilic carrier.

In some embodiments, the first lipophilic carrier has a melting point at least 5° C., at least 6° C., at least 7° C., at least 10° C., at least 12° C., or at least 15° C., higher than the second lipophilic carrier.

In some embodiments, the melting point of the composition is controlled by controlling the ratio of hydrogenated fats. In some embodiments, the release time of the probiotic bacteria, is controlled by the melting point of the composition. In some embodiments, the release time of the first agent, is controlled by the melting point of the composition. In some embodiments, the release time of the second, is controlled by the melting point of the composition.

In some embodiments, the release of the at least one probiotic bacteria in the form of biofilm is slower than the release of the second agent.

In some embodiments, the first lipophilic carrier and the second lipophilic carrier comprise cacao butter, palm oil, plant wax, vegetable wax, or any combination thereof.

In some embodiments, the first lipophilic carrier and the second lipophilic carrier comprise fatty acids derived from raw materials of vegetable origin.

In some embodiments, excipients are obtained by the esterification of fatty acids with alcohols such as glycerol, polyglycerol, propylene glycol and polyethylene glycol, and by the alcoholysis of vegetable oils and fats with glycerol, polyethylene glycol and propylene glycol.

### Agents

In some embodiments, the composition comprises probiotic bacteria in the form of biofilm a lipophilic carrier, and a first agent, in the form of a first layer. In some embodiments, the composition further comprises a second layer comprising a second agent.

In some embodiments, any one of the first agent and the second agent is an agent that improves the receptiveness of the vaginal tissue for colonizing probiotic bacteria. For example, an agent that may improve the receptiveness of the vaginal tissue for colonizing probiotic bacteria may be a pH modifier. In such case the lipophilic carrier is used to release an amount of a pH modifier that is sufficient to decrease the local pH in the vaginal tissue. Preferably, vaginal pH should be modified to about 4 which is optimal for colonization of the probiotic bacteria of the invention. In some embodiments, the pH modifier can be synthetic. In some embodiments the pH modifier can be natural-biological

In some embodiments, any one of the first agent and the second agent is a pH adjusting agent. In some embodiments, any one of the first agent and the second agent is a pH adjusting agent capable of adjusting the pH to 4.

Non-limiting examples of pH adjusting agents according to the present invention are sodium bicarbonate, ascorbic acid, citric acid, acetic acid, fumaric acid, propionic acid, malic acid, succinic acid, gluconic acid, tartaric acid, lactic acid, boric acid and cranberry extract.

In some embodiments, any one of the first agent and the second agent is an antibiotic.

In some embodiments, the antibiotic is any antibiotic used for treatment of bacterial vaginosis. Non-limiting examples of antibiotics include metronidazole (Flagyl), clindamycin (Cleocin), and metronidazole.

In some embodiments, the antibiotic is released first. In some embodiments, the probiotic bacteria is released after release of the antibiotic.

In some embodiments, the composition further comprises a stabilizer, a preservative, a lubricant, a viscosity modifying agent, a buffering agent, fatty acids, and combinations thereof.

One of skill in the art will appreciate that the order of the carriers and agents may be altered in various embodiments and that the nomenclature “first lipophilic carrier”, “first agent” and “second lipophilic carrier”, “second agent” is used herein for ease of reference. For instance, in some embodiments the second agent can be selected to be mixed with the one or more lipophilic carriers and the probiotic bacteria in the form of biofilm in the first layer. One of skill in the art will further appreciate that various systems may comprise more than two lipophilic carriers or agents.

### Particles

In some embodiments, the probiotic bacteria in the form of biofilm is attached to a particle.

In some embodiments, the average diameter of the particles is in the range of 50 micrometers to 1,500 micrometers (μm). In some embodiments, average diameter of the particles is in the range of 50 μm to 1,200 μm, 50 μm to 1,100 μm, 50 μm to 1,000 μm, 55 μm to 1,200 μm, 55 μm to 1,000 μm, 57 μm to 1,200 μm, or 60 μm to 1000 μm, including any range therebetween. Each possibility represents a separate embodiment of the invention.

In some embodiments, the particle is selected from: MCC, dicalcium phosphate (DCP), seeds, a polysaccharide, or any combination thereof.

In some embodiments, a seed is selected from: cranberries, passionfruit, herbals, oat, or any combination thereof.

As used herein, the term “polysaccharide” encompasses any polymeric of carbohydrates, made from monomeric monosaccharide that are linked to one another via a glycosidic bond.

In some embodiments, the polysaccharide comprises or consists of alginate.

In some embodiments, the particle comprises or consists of a food grade particle.

In some embodiments, food grade particle comprises or consists of a polysaccharide, a fat crystal, a protein, or any combination thereof.

In some embodiments, a food grade particle comprising a fat crystal is selected from: glycerol monooleate, glyceryl stearyl citrate, or a combination thereof.

In some embodiments, a food grade particle comprising polysaccharide is selected from: corn starch, starch nanocrystals, cellulose nanocrystals, microcrystalline cellulose, nano- or methyl cellulose, chitin, chitosan, or any combination thereof.

In some embodiments, a food grade particle comprising a protein is selected from: β-lactoglobulin, lactoferrin, lactoferrin-polysaccharide, bovine serum albumin, gelatin, soy protein isolate, pea protein, Zein, or any combination thereof.

In some embodiments, the food grade particle is selected from: flavonoid (tiliroside), wax, shellac-xanthan gum, or any combination thereof.

According to some embodiments, the particles in the composition described herein are adapted, configured or suitable for biofilm formation.

In some embodiments, the composition is formulated for vaginal administration.

In some embodiments, the composition is formulated for rectal administration. In some embodiments, the composition is formulated for vaginal administration and rectal administration.

### Use of the Composition

In some embodiments, the composition is adapted to colonize a vagina of a subject in need thereof. In some embodiments, the composition is adapted to colonize a rectum in a subject in need thereof.

In some embodiments, the composition is for use in the treatment of vaginosis, e.g., bacterial vaginosis.

In some embodiments, the composition is for use in treating or preventing a urogenital infection, dysbiosis, or both.

In some embodiments, the composition is for use in treating or preventing a dysbiosis related condition or disease.

As used herein, the term “dysbiosis related condition or disease” refers to any disease or a condition or a symptom associated therewith, which is characterized by imbalance of the microbial flora of an organism's tissue or body.

In some embodiments, the dysbiosis related condition or disease is selected from: bacterial vaginosis, a urogenital infection, ulcerative colitis, inflammatory bowel disease (IBD), and Crohn's disease.

In some embodiments, the composition is for use in treating or preventing yeast vaginitis, viral infection, fungal infection, bacterial vaginosis, urinary tract infection, or any combination thereof, in subject in need thereof.

In some embodiments, the composition is for use in modifying bacterial composition, or restoring the native vaginal flora, gut flora, or both, in a target site of a subject in need thereof.

In some embodiments, modifying bacterial composition in a subject refers to reduction or elimination of an unwanted bacteria, in the subject.

In some embodiments, the at least one probiotic bacteria in the form of biofilm is personalized for the subject.

In some embodiments, the composition is determined or prepared according to the profile of the subject to be treated (e.g., personalized treatment).

In some embodiments, the composition comprises one or more strains selected from Lactobacillus plantarum, Lactobacillus paracasei, Lactobacillus acidophilus, Lactobacillus delbrueckii subsp. Bulgaricus, Bifidobacterium breve, Bifidobacterium longum, and Faecalibacterium prausnitzii.

In some embodiments, the composition is an anti-inflammatory composition comprising one or more strains selected from Lactobacillus plantarum, Lactobacillus paracasei, Lactobacillus acidophilus, Lactobacillus delbrueckii subsp. Bulgaricus, Bifidobacterium breve, Bifidobacterium longum, and Faecalibacterium prausnitzii.

In some embodiments, the composition is formulated for vaginal administration.

In some embodiments, the composition is formulated for rectal administration.

In some embodiments, the composition is provided in a form of suppository.

In some embodiments, the composition is provided in a form of vaginal suppository, cream, tablet, capsule, ointment, gel or microcapsule.

In some embodiments, the composition can be administered for treating a medical condition associated with any disease, medical condition, or disorder as described herein throughout in a subject in need thereof.

In some embodiments, the treatment is combined with antibiotics treatment.

In some embodiments, the treatment is prophylactic, i.e., after antibiotic treatment.

In some embodiments, the vaginal tissue is pre-treated with a colonization agent prior to administration of the suppositories, wherein the pre-treatment improves the receptiveness of the vaginal tissue for colonizing probiotic bacteria.

### The Method

According to some embodiments, there is provided a method for preventing or treating a dysbiosis related condition or disease in a subject in need thereof, comprising administering to the subject a therapeutically effective amount of the herein discloses composition, thereby preventing or treating a dysbiosis related condition or disease in a subject.

According to some embodiments, there is provided a method for restoring the native flora in a subject in need thereof, comprising administering to the subject a therapeutically effective amount of the herein disclosed composition, thereby restoring the native flora in the subject.

In some embodiments, the subject is afflicted with or at risk of developing bacterial vaginosis, a urogenital infection, dysbiosis, ulcerative colitis, an inflammatory bowel disease (IBD), Crohn's disease, or any combination thereof.

According to some embodiments, the present invention provides a method for treating or reducing the risk of urogenital infections, dysbiosis, or both, in a subject, comprising administering an effective amount of a composition as described herein to the subject.

According to some embodiments, the present invention provides a method for treating or reducing the risk of ulcerative colitis, inflammatory bowel disease (IBD), Crohn's disease, or any combination thereof, in a subject, comprising administering an effective amount of a composition as described herein to the subject.

In some embodiments, the release of the at least one probiotic bacteria in the form of biofilm is controlled by the lipophilic carrier and the agent.

In some embodiments, the release of the at least one probiotic bacteria in the form of biofilm is controlled by the melting temperature of the lipophilic carrier. In some embodiments, different mixtures of lipophilic carriers can be used in order to tune the melting temperature of the composition.

### Method of Production

According to some embodiments, the present invention provides a process for producing a composition as described herein.

In some embodiments, a method for preparing the composition of the invention is provided, the method comprising the steps of: (a) inoculating a growth medium comprising a particle with a L. crispatus; (b) incubating the particle with L. crispatus from step (a) under conditions suitable for allowing L. crispatus to attach to the particle; (c) inoculating the particle of step (b) with at least one additional bacterial species; and (d) culturing the inoculated particle of step (c) under conditions suitable for forming a biofilm comprising: L. crispatus and at least one additional bacterial species.

In some embodiments, the at least one additional bacterial species is selected from the group consisting of: L. jensenii, L. gasseri, and L. rhamnosus.

In some embodiments, a method for preparing the composition of the invention comprises the steps of:
inoculating a growth medium comprising a particle with a first bacteria selected from: (i) L. crispatus; (ii) Bif. adolescentis; or (iii) F. prausnitzii;
incubating the particle with the first bacteria under conditions suitable for allowing the first bacteria to attach to the particle;
inoculating the particle with at least one additional bacterial species; and
culturing the inoculated particle under conditions suitable for forming a biofilm comprising: (i) L. crispatus; (ii) Bif. adolescentis; or (iii) F. prausnitzii, and at least one additional bacterial species.

In some embodiments, when the first bacteria is L. crispatus the at least one additional bacterial species is selected from: L. jensenii, L. gasseri, and L. rhamnosus.

In some embodiments, when the first bacteria is Bif. adolescentis the at least one additional bacterial species is selected from the group consisting of: Bif. Longum sub.longum, and Bif. breve.

In some embodiments, when the first bacteria is F. prausnitzii the at least one additional bacterial species is selected from the group consisting of: Bl. obeum, Bl. coccoides. Bac. vulgatus, and Dorea (Eubacterium) formicigenerans.

In some embodiments, when the first bacteria is F. prausnitzii the inoculating steps are performed simultaneously. In some embodiments, simultaneously refers to that the particle is inoculated with F. prausnitzii and the at least one additional bacteria at the same time. In some embodiments, simultaneously refers to that the particle is inoculated with F. prausnitzii and the at least one additional bacteria with no additional step therebetween. In some embodiments, simultaneously refers to that the particle is inoculated with F. prausnitzii and the at least one additional bacteria with no incubation time therebetween.

In some embodiments, the present invention provides a method or a process for producing a composition as described herein, comprising the steps of (i) mixing at least one probiotic bacteria in the form of biofilm with a first lipophilic carrier, and optionally a first agent, thereby forming a mixture and (ii) heating the mixture to a first heating temperature.

In some embodiments, the process further comprises the step of (iii) adding a second lipophilic carrier and a second agent.

In some embodiments, the ratio of the at least one probiotic bacteria in the form of biofilm and the first lipophilic carrier, is in the range of 1:1 to 1:10, 1:2 to 1:10, 1:5 to 1:10, 1:1 to 1:9, 1:1 to 1:5, including any range therebetween.

In some embodiments, the ratio of the at least one probiotic bacteria in the form of biofilm and the first agent, is in the range of 1:0.1 to 10:1, 1:0.5 to 10:1, 1:1 to 10:1, 1:2 to 10:1, 1:0.1 to 9:1, 1:0.1 to 8:1, 1:0.1 to 1:1, 1:0.1 to 2:1, including any range therebetween.

In some embodiments, the first lipophilic carrier and the second lipophilic carrier comprise one or more fatty acids with a saturated content of more than 40%, more than 41%, more than 45%, more than 48%, or more than 50%, including any value therebetween.

In some embodiments, the first lipophilic carrier and the second lipophilic carrier comprise one or more hydrogenated fats.

In some embodiments, the heating temperature is determined according to the melting temperature of the one or more hydrogenated fats.

### General

As used herein the term “about” refers to ±10%.

The terms “comprises”, “comprising”, “includes”, “including”, “having” and their conjugates mean “including but not limited to”.

The term “consisting of means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Generally, the nomenclature used herein, and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds.) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, New York (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference. Other general references are provided throughout this document.

### Materials and Methods

### Strains and Culture Conditions

All strains used in this study were purchase from either ATCC or DSMZ. Strains used in this study were: Lactobacillus crispatus (DSM 20584), Lactobacillus jensenii (DSM 20557), Lactobacillus gasseri (DSM), Lactobacillus rhamnosus (DSM/ATCC), Bifidobacterium longum sub. longum, Bifidobacterium breve, Bifidobacterium adolescentis, Faecalibacterium prausnitzii, Blautia obeum, Blautia coccoides, Bacteroides vulgatus, Dorea (Eubacterium) formicigenerans, Bacteroides uniformis, Ruminococcus gnavus, Blautia producta, Clostridium leptum, Clostridium (Blautia) coccoides, and Blautia (Ruminococcus) obeum.

L. gasseri and L. rhamnosus were aerobically grown in Animal-based medium (Himedia) or a Nonanimal-based growth medium suited for industrial production (NuCel by Procelys, France).

L. jensengfii and L. crispatus were grown anaerobically (90% N₂, 5% CO₂, 5% H₂ atmosphere). Anaerobic experiments were performed in the Bactron anaerobic workstation.

### Planktonic Cultures

Prior to the bacteria in form of biofilm resistance assays, planktonic bacteria resistant to acidity and antibiotics were determined. For low pH assay, an overnight culture of vaginal bacteria was diluted to achieve 10⁵-10⁹ colony forming units (CFU) per mL, based on the number of bacteria in biofilm of the specific strain. Planktonic bacteria were exposed to different acidity for 1 h at 37° C., similar to the procedure done for bacteria in form of biofilm. At the end of incubation, samples were centrifuges (max. speed, 2 min) and supernatant was removed. Bacteria pellet was then resuspended in phosphate buffered saline (PBS)×1 before plating and CFU counting.

For antibiotic assay, and overnight culture was diluted to an optical start density (OD) of 0.13. 100 μl of the culture was spread homogenously into agar plate and left for 15 min to dry prior to applying the antibiotic MIC strips (Himedia). Plates were incubated at 37° C. for 24 h before minimum inhibitory concentration (MIC) value were determined.

### Bacteria in Form of Biofilm Cultures

### Single Strain (Monoculture)

A culture of Lactobacillus sp. was started from glycerol stock (12.5%) and was incubated overnight at 37° C., 150-180 rpm at aerobically or anaerobically conditions, based on the specific strain. The culture was diluted to an OD600 of 0.05, for the final biofilm culture. The bacteria in form of biofilm cultures were obtained as described in WO2016181228A2, which is incorporated herein by reference in its entirety.

Bacteria in form of biofilm were grown at 37° C. with continuous stirring. All bacteria in form of biofilm experiments were carried out in laboratory-scale production either small-scale (volume of 30 mL) or medium-scale (volume of 250 mL or 500 mL in a fermenter of 2 L). Analysis of bacteria in form of biofilm growth was carried out every 24 h incubation during a total of 72 h. Each day, medium was replaced with a fresh medium and the number of bacteria in biofilm was quantified. Measurements of viable cells were done by counting CFU on agar plates. Additionally, bacteria in form of biofilm developmental stage was tested by exposing the bacteria in form of biofilm to extreme conditions such as low pH and antibiotics (for further details see section ‘pH and antibiotics resistance assays’). Results were latter compared to planktonic bacteria to demonstrate the advantage of bacteria in form of biofilm over free-living bacteria.

Few experiments were conducted to obtain the best conditions for bacteria in form of biofilm growth (following the methods described above):

Agitation—bacteria in form of biofilm growth and development (resistance to pH) was compared between static (no agitation) and continuous stirring conditions (70-80 rpm) or between two agitation speeds 70-80 rpm and 130 rpm (based on the experiment).

Incubation time—bacteria in form of biofilm growth and development (resistance to pH) was investigated each 24 h for a period of 72 h.

Particle sizes—bacteria in form of biofilm growth on different particles ranging between 80-1000 μM in size, was examined: Microcrystalline cellulose (MCC) (80-150 μM), Microcrystalline cellulose: Di calcium phosphate (MCC:DCP) (1:1, ˜100 μM), Cranberries (500-600 μM), Alginate (1,000 μM). Bacteria in form of biofilm was grown during 24 h and assessed for their growth development.

Matrix:Medium ratio—bacteria in form of biofilm growth during 24 h was evaluated as a function of ratio between matrix (MCC) to medium. The following ratio of matrix to medium were compared: matrix was either 2%, 5%, 10% or 20% from medium.

Agitation vs. no agitation after the addition of planktonic bacteria—the effect of mixing on the attachment of planktonic bacteria to the matrix, and subsequently bacteria in form of biofilm growth, was examined. Two conditions were compared following the addition of planktonic bacteria at the beginning of experiment: continuous agitation during the all experiment (24 h incubation) vs. no-agitation at the first 2 h (apart of two times 10 sec of gentle mixing during this time) and thereafter continuous agitation till the end of the experiment.

### pH and Antibiotics Resistance Assays

A sample from the media-matrix solution were transfer to tubes and then centrifuge at 500 rpm for 3 min at RT. Subsequently, the supernatant was discarded, and the pellet was washed with PBS to remove of planktonic bacteria that precipitated during centrifugation. Samples were centrifuge again at 500 rpm for 3 min at RT. Following centrifugation, supernatant was removed and for each treatment (pH or antibiotics) 1 g of bacteria in form of biofilm was used. For the pH resistance assay, bacteria in form of biofilm were exposed for 1 h to 5 mL PBS with different acidity: pH 2 and 3.5 (the pH of PBS×1 was adjusted to 2 and 3.5, respectively, using 2M HCl) for 1 h at 37° C., 100 rpm. Bacteria in form of biofilm incubated in 5 mL PBS 7 (ambient pH), with the same conditions, were applied as a control. For antibiotics assay, 1 gr of bacteria in form of biofilm were exposed to 3 sequential concentration of antibiotics, which were well above the MIC value of the specific strain. Bacteria in form of biofilm were incubated in 5 mL growth medium with or without antibiotic (the latter was used as a control) for 24 h at 37° C.

At the end of incubation, bacteria in form of biofilm were washed with 10 mL PBS x1. Following centrifugation (500 rpm, 3 min), 9 mL was removed and the bacteria in form of biofilm in the 1 mL remaining PBS solution were vortex for 1.5 min at high speed to release bacteria from biofilm that were attached to particles.

### Analytical Methods

CFU were determined at the end of each resistance assay. First, serial dilutions were conducted, and bacteria were plated in triplicate onto MRS agar plates. The plates were incubated at 37° C. in aerobic or anaerobic conditions, based on the strain growth condition (see above), for 48-72 h prior to CFU counting.

### Experimental Procedure

To optimize the bacteria growth two types of experimental designs were practiced, a small-scale experiment and a medium-scale experiment. Based on the results obtained from the small-scale experiment, the inventors define the conditions for the medium-scale experiment. The procedure in brief: the produced bacterial population growth and developmental state was examined every 24 hours of incubation during a total incubation time of 72 hours. Each day, medium was replaced with a fresh medium and the number of bacteria in biofilm was quantified (hereafter pH 7 treatment). Additionally, the formation and development of biofilm on the particles was tested by exposing the bacterial population to extreme conditions such as acidic pH (pH 3.5 and 2), a pH value close to the pH that prevails in a woman vagina (pH 4 5), and antibiotics (CB, carbenicillin; CIP, ciprofloxacin; VAN, vancomycin; NVB, novobiocin). pH resistance assay was conducted daily, and antibiotic assay was performed following 48 hours or 72 hours of incubation. Results from these assays were latter compared to planktonic bacteria of the same species to show the advantage of the produced bacterial population technologies over free-living bacteria. Additionally, to allow the bacteria to better attach to the matrix, on the first day after the addition of planktonic bacteria to the fermenter with the matrix, fermenter was kept in static conditions, for 2 hours at 37° C. (with a gentle mixing after 1 hour). A change of ˜1 log in bacteria yield was considered to be in the range of technical error of bacteria plating and CFU counting and was accounted for non-significant difference. The tested bacteria strains appear in Table 1.

### Produces a Bacterial Population

TABLE 1
Tested Bacteria strains

| Strain | Type | Strain number |
|---|---|---|
| Lactobacillus jensenii | Anaerobic | DSM 20557 |
| Lactobacillus crispatus | Anaerobic | DSM 20584 |
| Lactobacillus iners | Aerobic | DSM 13335 |
| Lactobacillus gasseri | Aerobic | ATCC 33323 |
| Lactobacillus rhamnosus | Aerobic | |

### Bacteria Strains Co-Culture

When two or more bacteria strains were co-cultured together, the strains were added separately to the particles:medium mixture with a suspension time between the addition of each bacterial strain to the mixture. After the addition of a bacterial strain to the mixture it was mixed well, and after 30 min to 1 hour another strain was added. This process was repeated if more bacteria strains were co-culture in the same mixture. The order of bacteria addition was decided based on anaerobic/aerobic strains or slow/fast-growing strains whereby the anaerobic or slow-growing strains were added before the aerobic or fast-growing strains.

### Mono-Cultures

### Single Strain

A culture of each bacteria strain that were used in the experiment was started from −80° C. stock (5% DMSO) and was incubated in anaerobic conditions overnight at 37° C. Unless otherwise stated, the culture was diluted to an OD600 of 0.05 (FaP, 0.03), for the final biofilm culture. The mono-culture in each experiment consist of growth medium (BfA, Bifidobacterium medium or Food-grade+casein; FaP, YCFAC or RCM), 20% mono-culture technologies particles (MCC:DCP, 1:1) and planktonic bacteria from the overnight culture. Mono-culture were grown at 37° C. with either continuous stirring conditions (small-scale, 80 rpm; fermentor, 300 rpm) or static conditions (only in small-scale). All the experiment was held in anaerobic glove box, however, the growth media for Bifidobacterium spp. was generally aerobic and for the experiments with Faecalibacterium was anaerobic. When continuous stirring was employed, the vessel was first left 5 h in static conditions to allow the bacteria to attach to the matrix. All mono-culture experiments were carried out in laboratory-scale production either small-scale (volume of 50 or 60 mL) or fermentation (volume of 3 L).

Analysis of mono-culture growth was carried out every ˜24 h incubation. Unless otherwise stated each day, medium was replaced with a fresh medium and the number of bacteria in biofilm was quantified. Measurements of viable cells were done by counting CFU on agar plates. In some cases, results were compared to planktonic bacteria to demonstrate the advantage of mono-culture technologies over free-living bacteria.

### Multi-Bacterial Combination

When few bacteria strains were co-culture together, the strains were added in parallel at initial OD of 0.03 or 0.05 for each strain. When time was the tested parameter, the slow-growing bacteria was added 24 h before the other species in the cluster. When OD was the tested parameter in the experiment, the slow-growing bacteria was added 10 times more than the other bacteria species in the cluster (initial OD 0.5). All other experimental setup and analysis was done as was described for single strain.

### Bacteria Quantification by Drop Assay

A sample from the media-matrix solution were transfer to tubes and then centrifuge at 500 rpm for 3 min at RT. Subsequently, the supernatant was discarded, and the pellet was washed with PBS to remove of planktonic bacteria that precipitated during centrifugation. Samples were centrifuge again at 500 rpm for 3 min at RT. Following centrifugation, supernatant was removed and for each treatment (pH or antibiotics) 1 g of sample was used. A third wash was done with 10 mL PBS×1. Following centrifugation (500 rpm, 3 min), 9 mL was removed and the biofilm in the 1 mL remaining PBS solution were vortex for 1.5 min at high speed to release bacteria from biofilm that were attached to particles.

### Analytical Methods

To determine the CFU, serial dilutions were conducted, and bacteria were plated in triplicate onto specific agar plate. The plates were incubated at 37° C. in anaerobic conditions, based on the strain growth condition (see above), for 48-72 h prior to CFU counting.

Coculture samples were sent to NGS analysis in order to obtain the relative abundance of each bacteria strain in the coculture. When it was applicable, relative abundance results were normalized to bacteria counts (CFU/mL) by the following equation:

“RA % of each bacterium*total bacterial count=specific species bacterium count”.

### EXAMPLE 1

### Suppositories Formulation

The formulation of the suppositories consists of bacteria in form of biofilm grown as described above, mixed in pharmaceutically acceptable excipients (oil-based carriers) and/or a prebiotic agent (such as, cranberries, ascorbic acid and antibiotics). Bacteria in form of biofilm used was either lyophilized (dry) or wet bacteria in form of biofilm. In the suppository formulation, vegetable (palm) butter and cocoa butter, in a ratio of 1:5, respectively, were melted in a hot bath at 50-52° C.

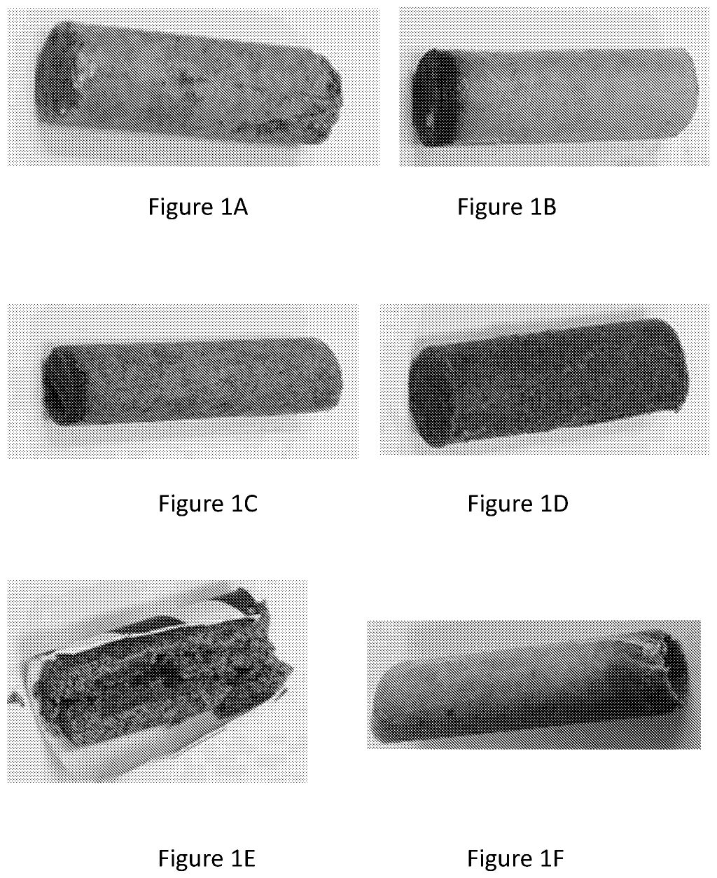

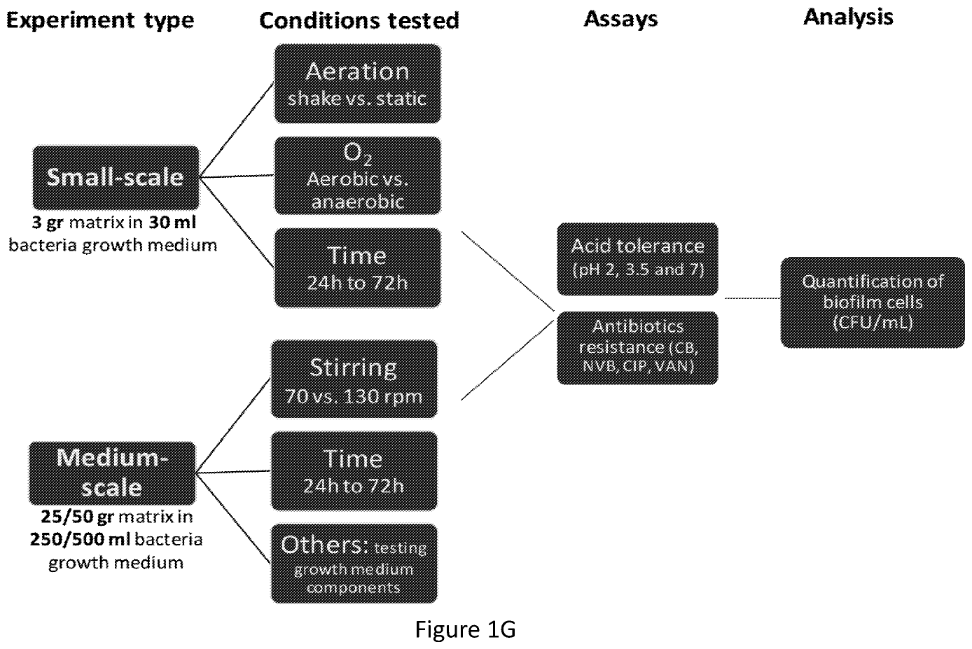

Temperature was monitored closely to not reach over a temperature that will compromise the butters (over 60° C.). Two to three drops of Lecithin were then added to the molten butters to aid with the homogeneity of the mixture. The mixture was left to cool down to 25° C. before bacteria in form of biofilm (dry) and/or prebiotic substance (such as cranberries, ascorbic acids etc.) were added in different rations of bacteria to wax. (Bacteria in form of biofilm and prebiotic) to butters, respectively. This final composition was reheated to 30° C. and was poured into vaginal suppositories molds. The suppositories were stored at 4° C. till use. To mimic the dissolution of the suppository, the suppositories were put at 37° C. in an incubator. Results are summarized in Table 2 and FIGS. 1A-1F.

TABLE 2

| Suppositories | | | | | | | Melting | % active in |
|---|---|---|---|---|---|---|---|---|
| formulation: | Oil | MCC | | Cranberries | | Illustration | time | suppository |
| 2 | Kahlwax 6240 | 700 | mg | 300 | mg | FIG. 1A | 130 min | 20% |
| | 4 g | | | | | | | |
| 3 | cocoa butter | 0.15 | g | 0.1 | g | FIG. 1B | 15 min | 5% |
| | 3.8 g | | | | | | | |
| | palm butter | | | | | | | |
| | 0.95 g | | | | | | | |
| 4 | cocoa butter | 700 | mg | 300 | mg | FIG. 1C | 20 min | 20% |
| | 4 g | | | | | | | |
| | palm butter | | | | | | | |
| | 1 g | | | | | | | |
| 5 | cocoa butter | 1.4 | g | 600 | mg | FIG. 1D | 40 min | 32% |
| | 2.4 g | | | | | | | |
| | palm butter | | | | | | | |
| | 0.6 g | | | | | | | |
| 6 | cocoa butter | 2.1 | g | 900 | mg | FIG. 1E | disintigrate | 60% |
| | 1.6 g | | | | | | | |
| | palm butter | | | | | | | |
| | 0.4 g | | | | | | | |
| 7 | Kahlwax 6240 | 700 | mg | 300 | mg | FIG. 1F | 30 min | 20% |

2 g
cocoa butter
1.15 g
palm butter
0.85 g

### EXAMPLE 2

### Bacteria Growth Optimization

Experimental procedure: The application of bacteria in form of biofilm was tested with two types of experimental designs a small-scale followed by a medium-scale experiment (FIG. 1G). Based on the results obtained from the small-scale experiment, the inventors define the conditions for the medium-scale experiment. The procedure in brief: Bacteria in form of biofilm growth and developmental state was examined every 24 h incubation during a total of 72 h. Each day, medium was replaced with a fresh medium and the number of bacteria in biofilm was quantified (hereafter pH 7 treatment). Additionally, the formation and development of biofilm on the particles was tested by exposing the Bacteria in form of biofilm to extreme conditions such as acidic pH (pH 3.5 and 2) and antibiotics (CB, carbenicillin; CIP, ciprofloxacin; VAN, vancomycin; NVB, novobiocin). pH resistance assay was conducted daily, and antibiotic assay was performed following 48 h or 72 h of incubation. Results from these assays were latter compared to planktonic bacteria of the same species to show the advantage of Bacteria in form of biofilm over free-living bacteria.

For each bacteria strain tested, the results are presented as follows:
Planktonic growth;
Optimization of Bacteria in form of biofilm growth in a Small-scale set-up;
Optimization of Bacteria in form of biofilm growth in a Medium-scale set-up.

In the description of the results, a change of 1 log in bacteria yield is considered to be in the range of technical error of bacteria plating and CFU counting and thus is accounted for non-significant difference.

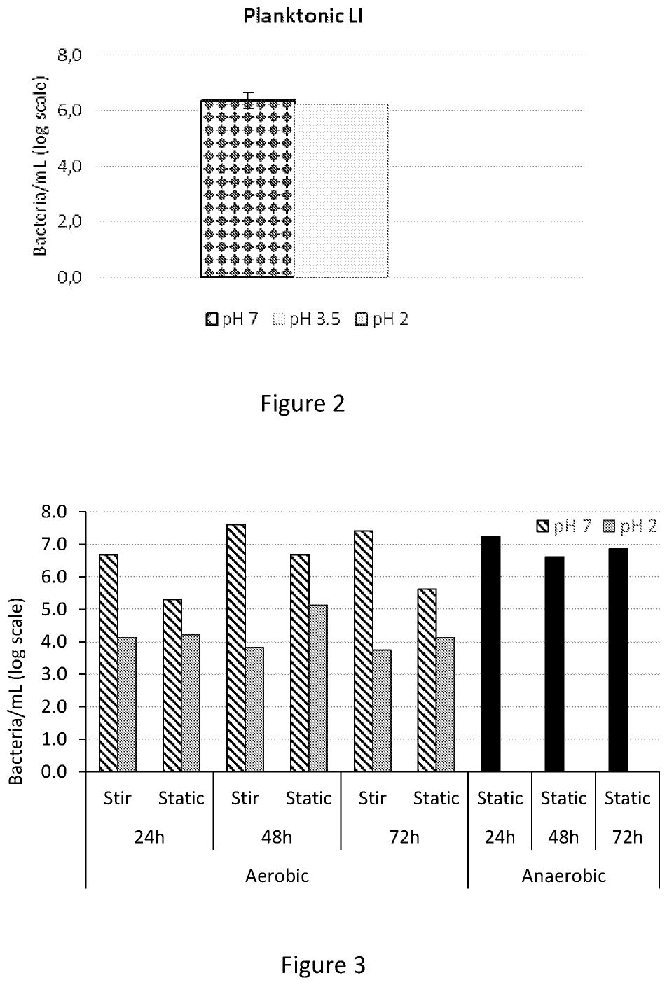

Planktonic bacteria produced moderate bacteria yield of ˜10⁶ cells/mL at pH 7 (control, FIG. 2). Bacteria yield at pH 3.5 was comparable to control. However, at higher acidic conditions (pH 2) planktonic bacteria did not survived. Finally, exposure to antibiotics showed a MIC of 64 μg/mL of CB and 16 μg/mL of NVB and 4 μg/mL to VAN (Table 3).

TABLE 3
MIC of different antibiotics for planktonic LI.
Values are expressed in μg/mL

| Bacteria\ABX | CB | NVB | VAN |
|---|---|---|---|
| Lactobacillus iners | 64 | 16 | 4 |

### Lactobacillus iners (LI) Bacteria in Form of Biofilm—Small-Scale Experiment

Results from the small-scale experiment showed the highest bacteria yield was after 48 h incubation with the matrix, in aerobic conditions with a gentle agitation (100 rpm). In contrast to planktonic bacteria, Bacteria in form of biofilm survived pH 2, with a drop of 1 to 3.8 log in bacteria growth in biofilm for agitated and non-agitated conditions, respectively (FIG. 3). However, in anaerobic conditions bacteria in biofilm did not show any advantage at low pH compared to planktonic bacteria.

### LI Bacteria in Form of Biofilm—Medium-Scale Experiment

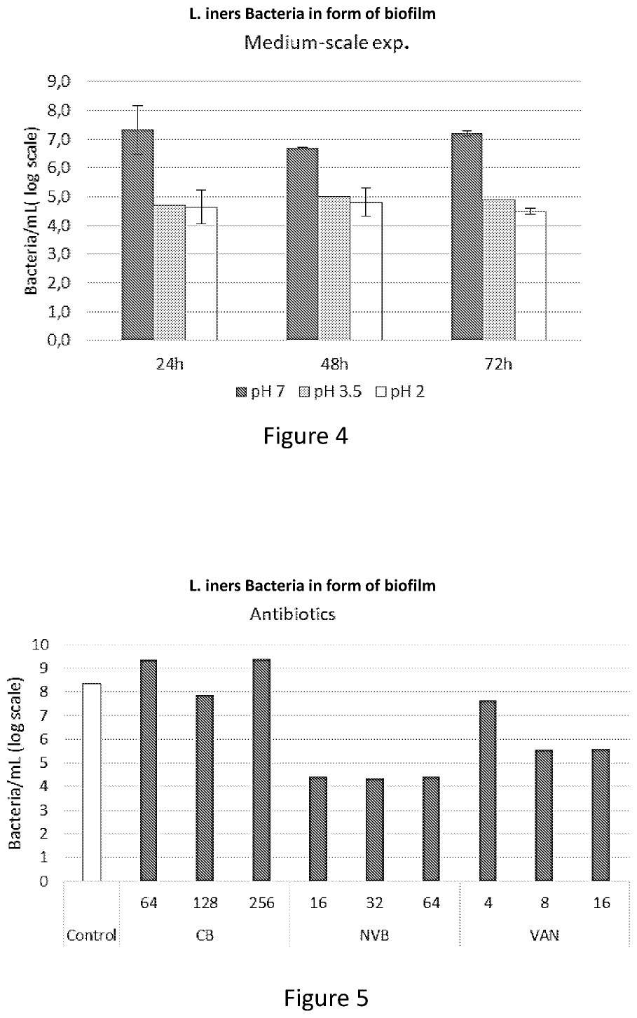

Based on results from the small-scale experiment, conditions employed for the growth of LI in biofilm were aerobic with a gentle agitation. Here, bacteria yield seems to be slightly higher after 24 h and 72 h incubation with the matrix compared to 48 h. Bacteria in form of biofilm survived at both acidic pH treatments and there were no significant differences in survival rate between treatments (FIG. 4).

Finally, Bacteria in form of biofilm was tested for resistance to antibiotics (FIG. 5). While Bacteria in form of biofilm demonstrated resistance to all three types of antibiotics compare to planktonic bacteria, the highest resistance was observed for the CB antibiotic. When exposed to CB, bacteria yield was either not affected or slightly affected by antibiotic concentration, even after 24 h incubation. Bacteria in form of biofilm exposure to VAN and NVB antibiotics showed a similar trend of bacteria growth between incubation days: after an initial decrease of ˜4 log, numbers of bacteria did not change significantly with increasing concentrations. When antibiotics resistance data are pooled together, it appears that after 48 h the inventors obtained the highest resistance of biofilm to increasing amount of antibiotics.

In conclusion, max bacteria yield in biofilm of L. iners, based on the applied experimental conditions, was 10⁷-10⁸. LI Bacteria in form of biofilm were able to survive and/or grow well in the presence of both acidic pH and antibiotics thus demonstrating that Bacteria in form of biofilm performance was superior to that of free-living bacteria.

### L. jensenii (LJ)

### Planktonic LJ

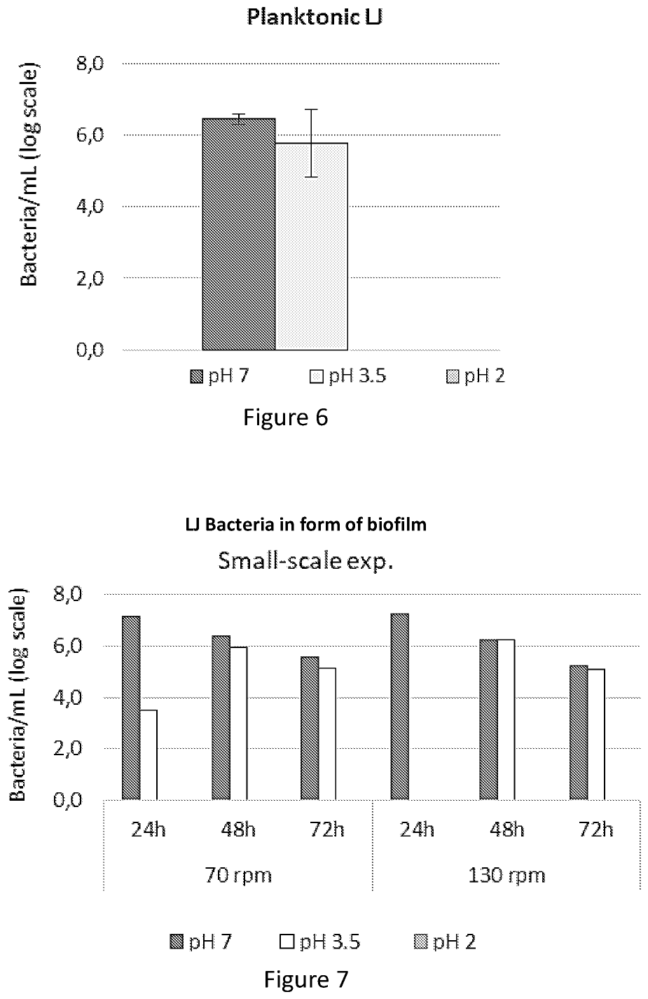

Planktonic bacteria produced bacteria yield of ˜10⁶ cells/mL at control conditions (pH 7, FIG. 6). No significant difference was observed in bacteria yield at pH 3.5 compared to control. However, planktonic bacteria did not survive exposure to pH 2.

Exposure of planktonic bacteria to CB and NVB and VAN antibiotics resulted in low bacterial resistance to antibiotic with a MIC of 8 μg/mL, 2 μg/mL and 1.5 μg/mL, respectively. However, planktonic bacteria were not susceptible to CIP and displayed full growth of bacteria cells regardless the employed antibiotic concertation (Table 4).

TABLE 4
MIC of different antibiotics for planktonic LT Values are expressed in
μg/mL

| Bacteria\ABX | CB | NVB | VAN | CIP |
|---|---|---|---|---|
| Lactobacillus jensenii | 8 | 2 | 1.5 | >256 |

### LJ Bacteria in Form of Biofilm—Small-Scale Experiment

Growth of LJ Bacteria in form of biofilm in the small-scale experimental set-up was similar at both agitation conditions (70 rpm and 130 rpm) (FIG. 7). There was a decrease over time in Bacteria in form of biofilm growth. Maximum bacteria yield of 10⁷ cells/mL was observed after 24 h of incubation while at the third day of incubation, the lowest bacteria yield (˜10⁵ cells/mL) was recorded. When Bacteria in form of biofilm were exposed to pH 3.5, excluding the 1^{st} day of incubation, there was no significant difference in bacteria number compare to control (pH 7). However, Bacteria in form of biofilm did not survive the lowest pH treatment (pH 2). In general, growth of bacteria in biofilm did not differ significantly from their free-living form. It should be noted that an experiment was performed to test whether no agitation can result in better yield of Bacteria in form of biofilm (RD206). Results showed a decrease of ˜1 log in bacteria growth compare to both agitation conditions.

In the following medium-scale experiments, both agitation conditions were tested as there was no definitive conclusion regarding which stirring condition have the best effect on Bacteria in form of biofilm growth. Furthermore, as the setup of medium-scale experiments resemble better the growth conditions utilize in the industry, it was decided to examine these two agitation speeds with this type of setup as well.

### LJ Bacteria in Form of Biofilm—Medium-Scale Experiment

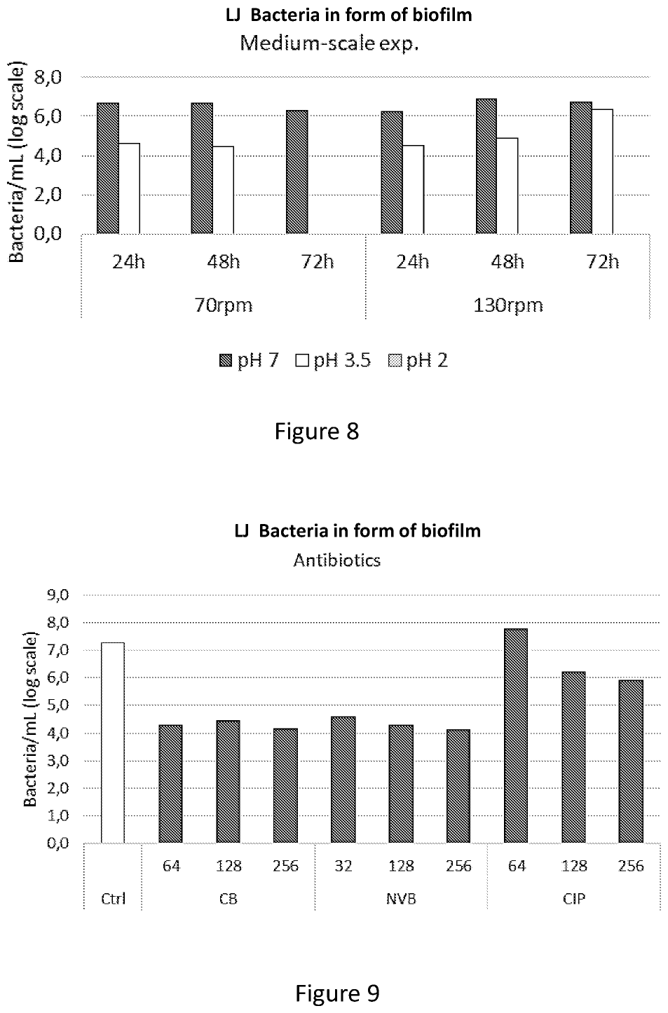

Similar to the small-scale experiment, bacteria yield in biofilm reached a maximum growth of ˜10⁷ CFU/mL, however, growth stayed stable during all 3 days of incubation (FIG. 8). Moreover, no significant difference was observed between the two agitation speeds.

Different from the former experiment, a larger decrease was observed in CFU counts after exposure to pH 3.5 (2-2.3 log at the end of the first two days of incubation (FIG. 8). When Bacteria in form of biofilm resistance to pH 2 was tested, biofilm completely disintegrated (FIG. 8). The inventors speculate that the disappearing of cells after incubation at pH 3.5 at the 3^{rd} day of incubation, was due to technical error. Indeed, in the following experiments, results at 72 h did not differ from the first two days of incubation.

Next, LJ Bacteria in form of biofilm was exposed to increase antibiotics concentrations (FIG. 9). Bacteria in form of biofilm exhibited high resistance to antibiotics compared to planktonic cells whereas the applied concentrations were well-above the MIC value for planktonic bacteria. For both NVB and CB, although after 24 h there was an initial reduction in bacteria growth, yield stayed stable regardless the antibiotic concentration.

In summary, despite Bacteria in form of biofilm results to acidic conditions, LJ Bacteria in form of biofilm demonstrated a clear advantage over planktonic bacteria when was exposed to antibiotics.

Although in the current experiment there was no difference in agitation conditions, experiments that were conducted latter, in medium-scale set-up, produced better results for Bacteria in form of biofilm growth at low agitation. Therefore, agitation for LJ Bacteria in form of biofilm in this type of experiments was set to continuous 70-80 rpm. Additionally, a preliminary experiment showed an advantage for Bacteria in form of biofilm growth when at the first day, after the addition of planktonic bacteria to the fermenter with the matrix, fermenter is kept in static conditions, for 2 h at 37° C. (with a gentle mixing after 1 h). This step may allow the bacteria to better attach to the matrix and was employed in all subsequent experiments, regardless the bacteria strains that is being used.

### L. Crispatus (LCr)

### Planktonic LCr

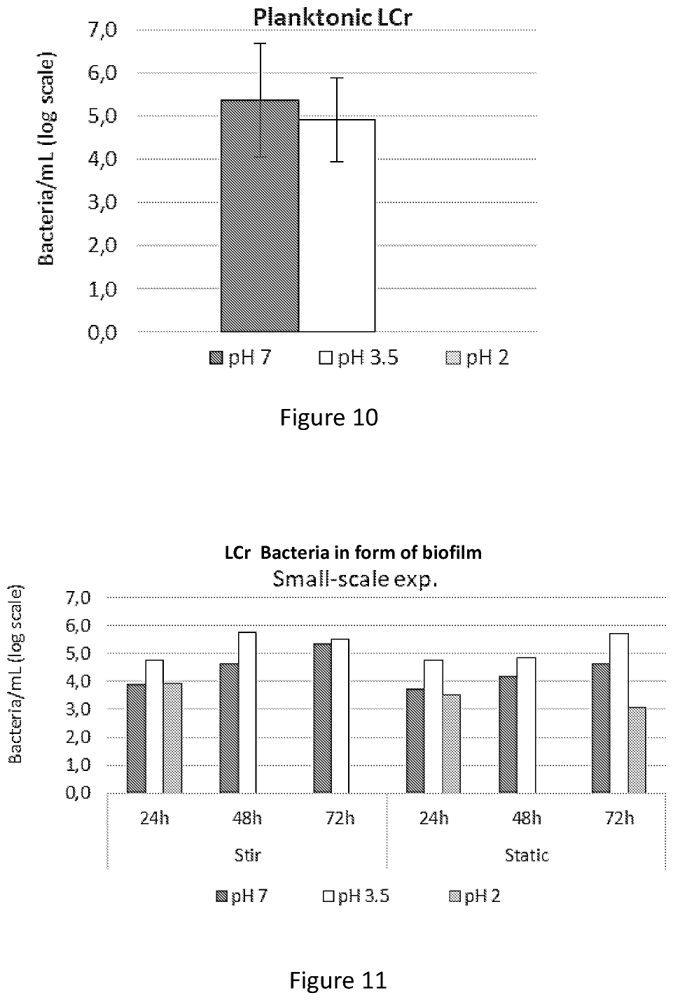

Planktonic LCr exhibited similar results to planktonic LJ when exposed to low pH treatments and increased antibiotic concentrations (FIG. 10). Exposure to pH 3.5 did not significantly affected bacteria cells compare to control (pH 7) whereas at pH 2 bacteria did not survive.

When planktonic LCr were treated with antibiotics, low bacteria resistance was observed for CB, NVB and VAN with MIC values of 4 μg/mL, 2 μg/mL and 1.5 μg/mL, respectively (Table 5). However, planktonic bacteria were not susceptible to CIP and displayed full growth of bacteria cells regardless the employed antibiotic concertation.

TABLE 5
MIC of different antibiotics for planktonic LCr
Values are expressed in μg/mL.

| Bacteria\ABX | CB | NVB | VAN | CIP |
|---|---|---|---|---|
| Lactobacillus crispatus | 4 | 2 | 1.5 | >256 |

### Bacteria in Form of Biofilm—Small-Scale Experiment

Whether LCr Bacteria in form of biofilm were regularly agitated or not agitated, small-scale experiment with LCr produced maximum bacteria yield of 5×10⁵ cells/mL and lowest of ˜10⁴ cells/mL (FIG. 11). In both treatments, Bacteria in form of biofilm growth was the highest after 3 days on incubation with the matrix. Surprisingly, under moderate acidic conditions (pH 3.5), an increase of ˜1-2 log in bacteria yield was observed regardless the treatment. Nonetheless, at pH 2 number of bacteria in biofilm either decreased by 2-4 log or completely diminished. No pattern could be determined in their survival at this acidic condition and their resistance to pH 2 will be re-examined in the following medium-scale experiment.

Despite the similarity in the results from both stirring conditions, Bacteria in form of biofilm growth when agitated appear to produce a slightly better growth and survival at pH 7 and 3.5, respectively. Gentle agitation (70-80 rpm) was therefore employed in the next experiments.

### LCr Bacteria in Form of Biofilm—Medium-Scale Experiment

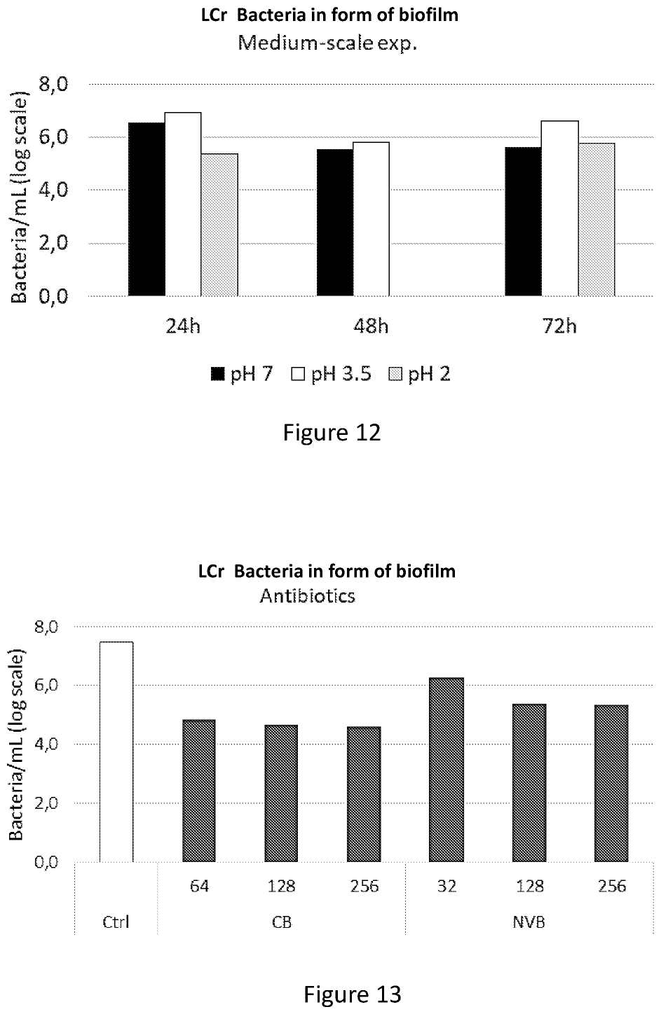

Bacteria yield in biofilm was slightly higher (˜1-2 log) at the medium-scale experiment compare with the small-scale experiment (FIG. 12). However, the increase in Bacteria in form of biofilm when exposure to pH 3.5 was comparable to the ones observed in the small-scale experimental set up. High survival of LCr Bacteria in form of biofilm was recorded after the 1^{st} and 3^{rd} of incubation, when exposed to the lowest pH treatment. In both experimental set-up, Bacteria in form of biofilm at this pH treatment, perished after 48 h.

Resistance of LCr Bacteria in form of biofilm to antibiotics was then investigated (FIG. 13). Similar to LJ Bacteria in form of biofilm, LCr Bacteria in form of biofilm showed high resistance to CB and NVB with a slightly better growth (1 log) of LCr Bacteria in form of biofilm after exposure to NOVO.

To summarize, LCr Bacteria in form of biofilm showed moderate advantage to low pH treatment over their planktonic counterpart and high advantage when tested against antibiotics. Moreover, number of biofilm cells after 24 h and 72 h of incubation and following exposure to low pH treatments were comparable.

### L. gasseri (LG)

### Planktonic LG

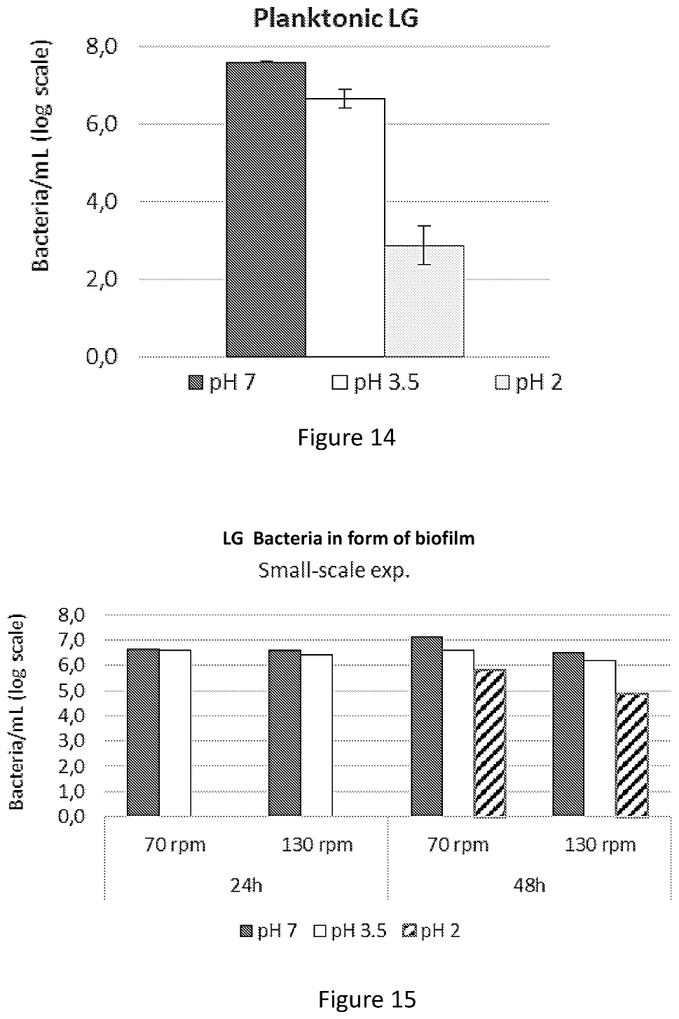

Results of planktonic LG, when exposed to acidic pH, differed from planktonic LI, LJ and LCr (FIG. 14). While exposure to pH 3.5 slightly decrease number of planktonic LG, incubation in pH 2 resulted in survival of bacteria in very low numbers.

When planktonic LG were later tested for their susceptibility to antibiotics (Table 6), MIC values were established; similar to LJ and LCr, planktonic LG were highly sensitive to for CB, NVB and VAN (4 μg/mL, 2 μg/mL and 1.5 μg/mL, respectively) while for CIP bacteria showed full resistance.

TABLE 6
MIC of different antibiotics for planktonic LG
Values are expressed in μg/mL.

| Bacteria\ABX | CB | NVB | VAN | CIP |
|---|---|---|---|---|
| Lactobacillus gasseri | 4 | 2 | 1.5 | >256 |

### LG Bacteria in Form of Biofilm—Small-Scale Experiment

Similar to LCr and LJ Bacteria in form of biofilm, no significant difference was observed between the two stirring speeds that were tested (FIG. 15). The highest bacteria yield was 10⁷ cells/mL. At 48 h, after exposure to acidic pH Bacteria in form of biofilm displayed high viability; Number of bacteria in biofilm did not differ between pH 3.5 to control whereas there was only a 1.3 and 1.6 log drop, at 70 rpm and 130 rpm respectively, in bacteria when inoculated in pH 2. This result indicates the performance of biofilm cells to be superior to that of planktonic cells, where in the latter cells completely perished at pH 2.

### LG Bacteria in Form of Biofilm—Medium-Scale Experiment

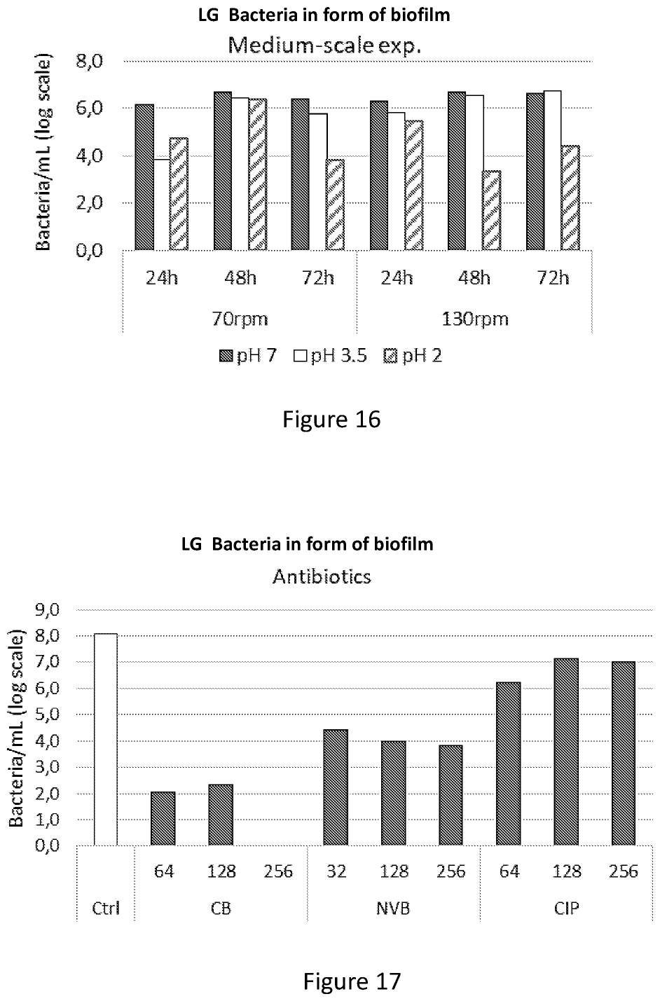

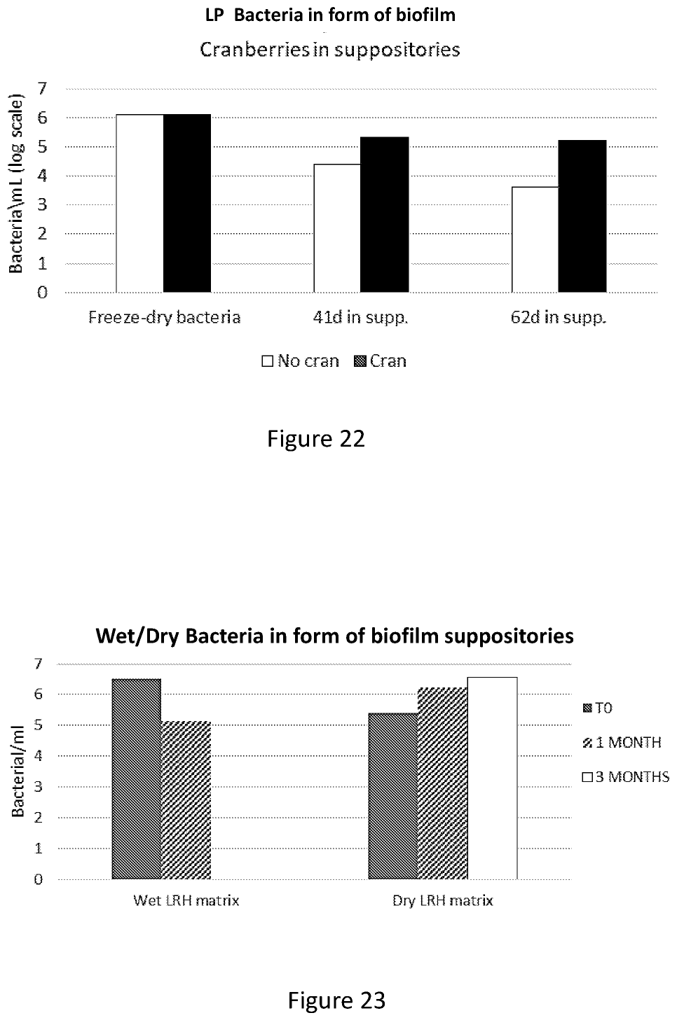

When the two agitation conditions where compared, no significant difference was detected in the number of biofilm-embedded bacteria cells (FIG. 16), regardless the incubation time. Furthermore, at both treatments, high survival of biofilm cells was observed, after exposure to the lowest pH treatment. The decrease in cells viability at this pH treatment was not more than 3.2 log (after 48 h of incubation at 130 rpm). The relatively large survival at pH 2, was observed also in other experiments (see FIG. 22-24). At pH 3.5, survival of LG Bacteria in form of biofilm appears to be slightly better when grown at 130 rpm compared to 70 rpm. However, at pH 2, no difference was detected in the survival of Bacteria in form of biofilm between the two agitation speeds.

In a later experiment, when similar stirring speeds were tested again, growth rate of LG Bacteria in form of biofilm were more enhanced at the lower speed (results are not shown). Thus, as for the former strains in this project, the mixing speed was chosen to be 70-80 rpm for the future experiments.

LG Bacteria in form of biofilm were able to survive and grow well in the presence of NVB antibiotic (FIG. 17). However, in the presence of CB, Bacteria in form of biofilm survival was less distinct with only few colonies that grow after exposure to this antibiotic. The number of survived colonies where below the threshold that was consider as significant (FIG. 17; Dashed line).

To conclude, LG planktonic and Bacteria in form of biofilm cultures showed large difference between the two modes of growth in relation to their resistance to extreme conditions. The advantage of LG Bacteria in form of biofilm over suspended bacteria is therefore evident.

### L. rhamnosus (LRh)

### Planktonic LRh

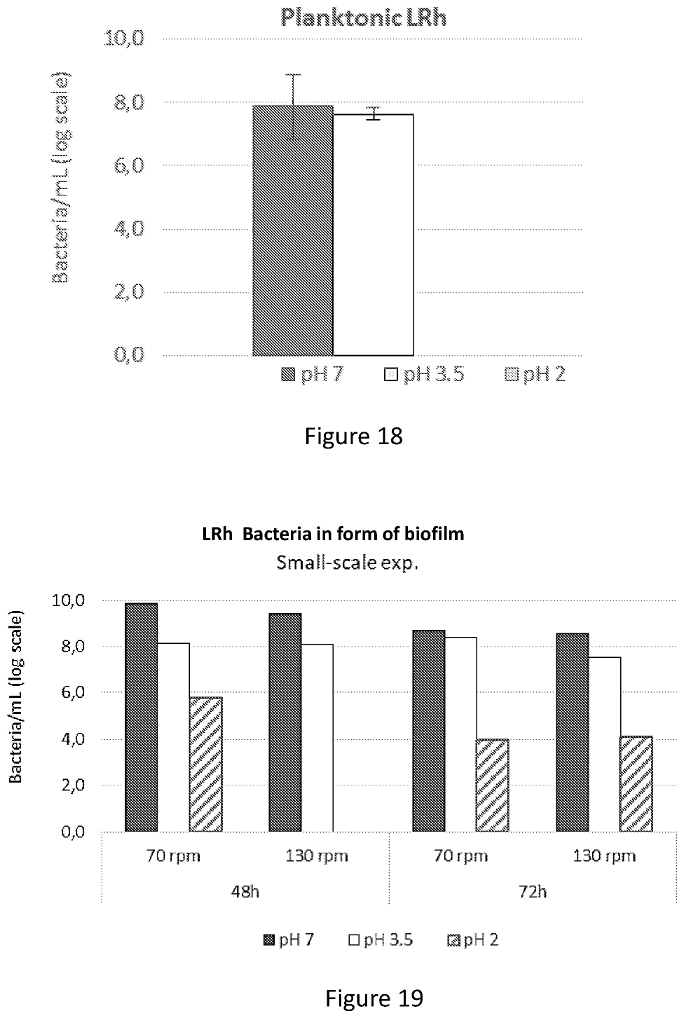

Planktonic LRh cells showed similar response to all bacteria strains that are described in this project, when exposed to increase acidity; no difference was detected between the control sample and Bacteria in form of biofilm exposed to pH 3.5 whereas cells disappeared at pH 2 (FIG. 18).

Similar response to the other lactobacillus sp. used in this project was also observed when suspended cells of LRh were exposed to CB and NVB antibiotics (MIC values of 4 and 2 μg/mL; Table 7). Nonetheless, when inoculated in the presence of CIP and VAN antibiotics, their response was opposite to the other bacteria strains; planktonic LRh were highly sensitive to CIP (0.25 μg/mL) and completely resistant to VAN (>256 μg/mL).

TABLE 7
MIC of different antibiotics for planktonic LRh
Values are expressed in μg/mL.

| Bacteria\ABX | CB | NVB | VAN | CIP |
|---|---|---|---|---|
| Lactobacillus rhamnosus | 4 | 2 | >256 | 2 |

### LRh Bacteria in Form of Biofilm—Small-Scale Experiment

Maximum number of biofilm-embedded bacteria was ˜10¹⁰ CFU/mL (FIG. 19). Overall, growth of LRh Bacteria in form of biofilm that have experienced 70 rpm seem to be slightly better than Bacteria in form of biofilm that have experienced 130 rpm; this was expressed in their high survival at pH 2 after 48 h (5.8 log) and their slightly improved survival at pH 3.5 (compare to pH 7) at the 3^{rd} day of incubation. In addition, at 72 h of incubation there is a small decrease in the number of biofilm-embedded bacteria.

Based on the results from this experiment, the employed agitation in the next experiments was ˜70-80 rpm.

### LRh Bacteria in Form of Biofilm—Medium-Scale Experiment

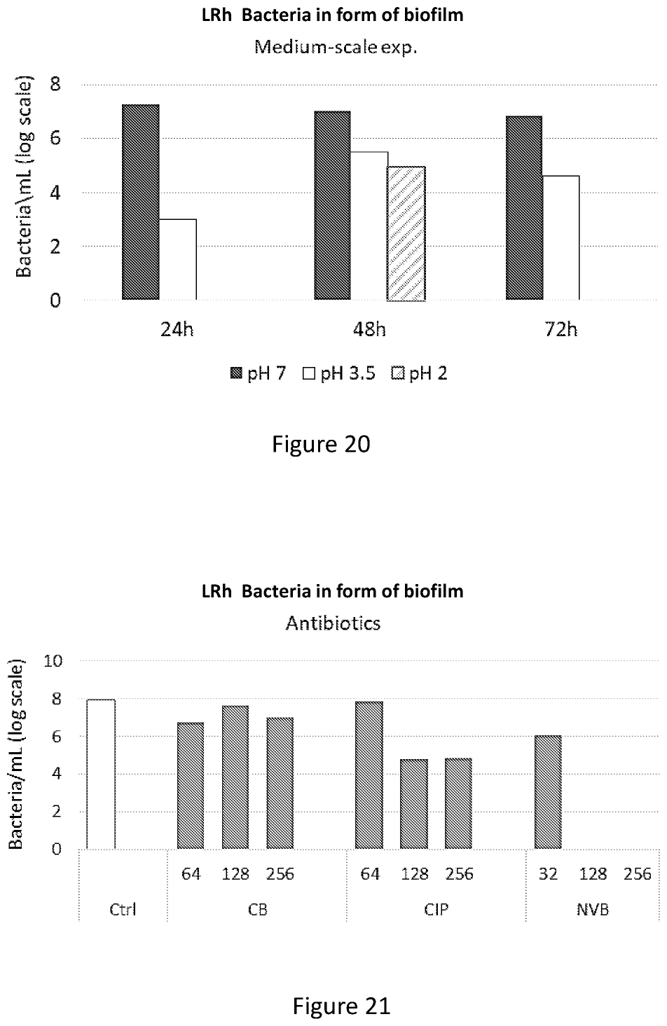

Unlike the former small-scale experiment, the average number of biofilm-embedded bacteria did not exceed 10⁷ CFU/mL (FIG. 20). This difference was due to an additional washing step that was included in the protocol to improve separation of the suspended bacteria from the Bacteria in form of biofilm. This washing step was then employed in all experimental designs from this point onwards. Over time, growth rate of LRh Bacteria in form of biofilm remained constant. However, in this experiment, LRh Bacteria in form of biofilm appear to better resist low pH treatments at the end of the second day of incubation; a reduction of not more than 2 logs was observed at pH 2.

In the presence of antibiotics, LRh Bacteria in form of biofilm survived and grew well when exposed to CB and CIP but did not survived the high concentrations of NVB (128 and 256 μg/mL; MIC, 2 μg/mL FIG. 21).

LRh Bacteria in form of biofilm have showed increased survived and growth in the presence of CB and CIP antibiotics, in concentrations where suspended planktonic counterparts completely disappeared.

### EXAMPLE 3

### Evaluation of Suppositorie Formulations

The formulation of the suppositories consists of Bacteria in form of biofilm, mixed in pharmaceutically acceptable excipients (oil-based carriers) and/or a supplement. Bacteria in form of biofilm was used either as lyophilized (dry) powder or as wet Bacteria in form of biofilm (at the end of 72 h incubation). A stability assay of the Bacteria in form of biofilm survival in suppositories was preformed once a month, for the duration of 6 months. Each month, one suppository from each formulation was melted in PBS (×1) and bacteria were plated for CFU counting (see Analytical methods).

### Improving Active Ingredients Mixture

Additives. High vaginal pH is associated with an increase of vaginal pathogens and the acidity of the vagina has long been understood to be a protective mechanism against colonization of anaerobes pathogens while creating a favourable environment for the lactobacilli to thrive. Examining the effect of different additives (cranberries and ascorbic acid) on Bacteria in form of biofilm LP (as part of a small scale). Here, the inventors examined two acidifying agents, cranberries and ascorbic acid, where the former is also suggested to have a role in preventing and/or reducing recurrent of UTI infection. The aim is to add one of these additives along with the Bacteria in form of biofilm in the suppository composition. As such, their effect on Bacteria in form of biofilm survival and growth was tested. Following exposure of LP Bacteria in form of biofilm to cranberries powder (300 mg), no significant effect on Bacteria in form of biofilm survival and/or growth was recorded (FIG. 27).

Interestingly, when cranberries powder was included in suppositories together with lyophilized bacteria in form of biofilm, an enhancement in bacterial survival was observed compared to when cranberries were omitted from the suppositories (between 1 to 1.6 log higher; FIG. 22). In addition, samples were stable during two months in suppositories (FIG. 22). The addition of Ascorbic acid (AA; vitamin C) to suppositories, produced similar results to the addition of cranberries; Only a small reduction of 1 to 2 log was observed in Bacteria in form of biofilm growth. As expected, both AA and cranberries reduced initial pH values in the MRS solution to 3.5-4. Because the addition of AA provided a more homogeneous mixture for the suppositories, it was decided to use it in later experiments.

Bacteria in form of biofilm. The survival of wet Bacteria in form of biofilm (after 72 h incubation) and dry bacteria in form of biofilm in suppositories was investigated. Results revealed that after 1 month in suppositories CFU counts of dry Bacteria in form of biofilm did not differ significantly from their number at T0 (less than 1 log). However, there was a reduction of 1.5 log in wet Bacteria in form of biofilm survival. After 3 months, however, wet Bacteria in form of biofilm of LRh completely perished while CFU count of dry Bacteria in form of biofilm in suppository remained stable. This result clearly demonstrates that humidity negatively affected Bacteria in form of biofilm survival in suppositories, and therefore the use of dry Bacteria in form of biofilm powder is essential (FIG. 23).

### Improving Suppositories Excipients Mixture

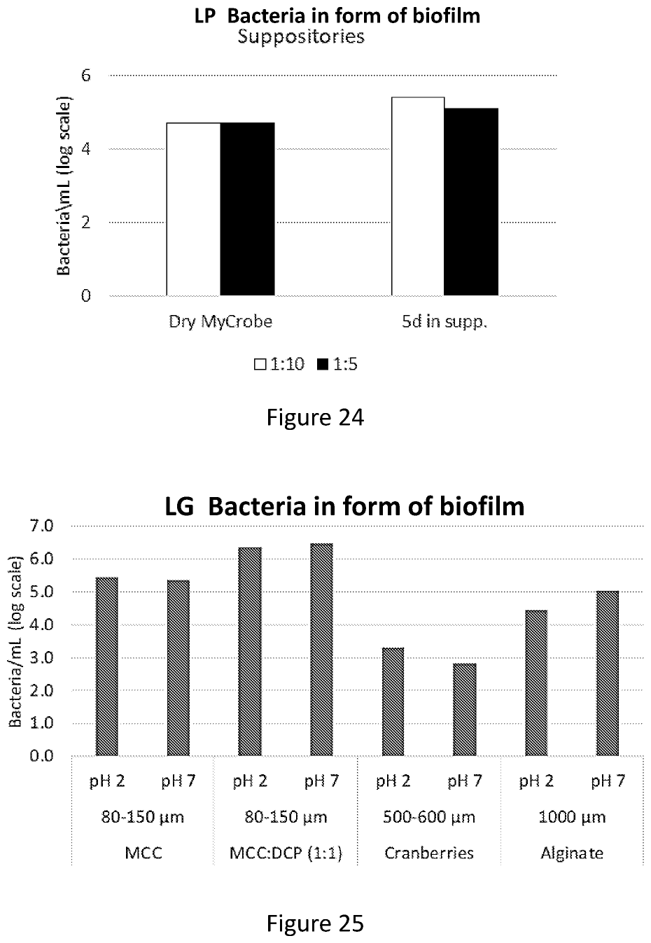

After few small experiments, the basic formulation of the oil-based carriers included vegetable (palm) butter and cocoa butter, in a ratio of 1:5, respectively as well as few drops of Lecithin to aid with the homogeneity of the mixture. Next, the inventors aimed to reduce the volume of oil-based carriers compare to bacteria in form of biofilm, thus increasing the quantity of bacteria in form of biofilm in the suppositories. Two ratios of bacteria in form of biofilm to carriers were tested, 1:5 and 1:10, respectively (FIG. 24). Results showed no difference in Bacteria in form of biofilm growth between the two ratios, thus allowing us to raise Bacteria in form of biofilm quantity in the suppositories composition.

The addition of a supplement such as cranberries or vitamin C did not affect Bacteria in form of biofilm growth and their administration together with Bacteria in form of biofilm can be more effective for treating BV than the administration of each one alone.

### Particle Sizes and Bacteria in Form of Biofilm Affinity to the Particles

After 24 h incubation of LG planktonic cells with different particles, LG Bacteria in form of biofilm growth and development was as follows Microcrystalline cellulose: Di calcium phosphate (MCC:DCP)>MCC>Alginate>Cranberries. Based on these results the inventors suggest that the combination of both reduced particle size and the type of matrix positively influenced LG Bacteria in form of biofilm growth and development: (1) particle sizes—smaller-particles size (higher surface to volume ratio) may allow more bacteria to attach per particle volume, thus improving Bacteria in form of biofilm growth: Bacteria in form of biofilm growth was enhanced on MCC or MCC:DCP combination (80-150 μm) compare to alginate beads (1,000 μm); (2) type of matrix—the inventors suggest here two possible explanation, without wishing to be bound to any particular theory, for the contribution of the specific matrix to biofilm growth and formation. Despite alginate being twice as big as cranberries, LG Bacteria in form of biofilm growth was higher when inoculated with alginate. One assumption is the lower pH that is induced by the presence of cranberries in the solution, which might negatively have affected Bacteria in form of biofilm growth. Another speculation is based to the fact that alginate is one of bacterial polysaccharides that was shown to be important for biofilm formation. Another example is the difference in the number of LG biofilms cells between MCC to MCC:DCP. Few studies have shown that exogenous Calcium ions can promote biofilm formation, hence the presence of soluble DCP particles (dicalcium phosphate) and subsequently calcium ions may enhance growth and development of LG biofilm.

### EXAMPLE 4

### Suppositories Formulations

A new combination of bacteria with Pentasa (anti-inflammatory agent) as rectal/vaginal suppositories, is shown.

Two formulations were compared, a formulation A with dry Bacteria in form of biofilm, and a formulation B, with a combination of Pentasa and Bacteria in form of biofilm (Table 8).

TABLE 8

| | Amount | Oil/fatty | Dry Bacteria | Medicine |
|---|---|---|---|---|
| A | 3 units | 8 g | LG, LGG | — |
| | | | 0.5 g each | |
| B | 3 units | 8 g | LGG, LG 0.66 g each | Pentasa 0.66 g |

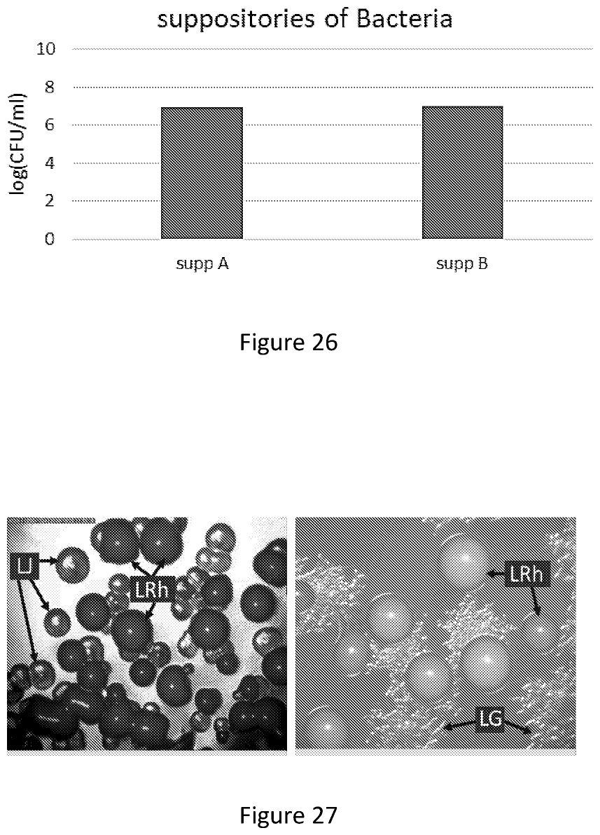

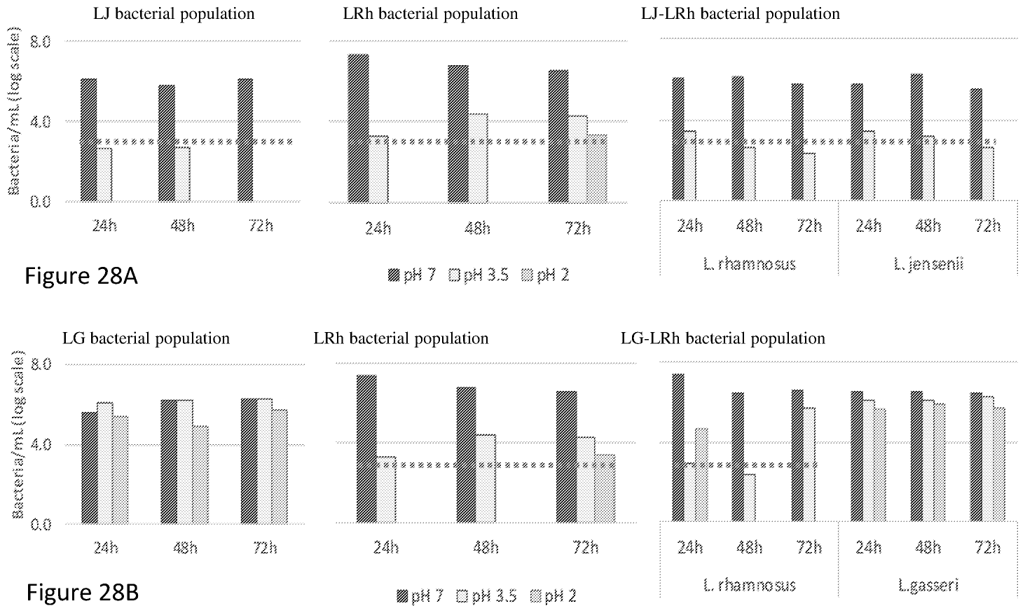

When tested the combination of Pentasa with dry Bacteria in the form of biofilm, the stability of the bacteria maintained (FIG. 26). The same was observed for planktonic bacteria with Pentasa.

### EXAMPLE 5

### Co-Cultured Biofilm

Co-Culture with Two Bacterial Strains

LJ and LG were identified as anaerobic/slow-growing bacteria whereas LRh was identified as an aerobic/fast-growing bacteria. When LJ or LG planktonic cells were co-inoculated with LRh, they were added prior to LRh with a gap of about 1 hour between the additions of the strains. No significant difference was recorded in biofilm growth (max. of 1 log) and development (resistance to pH), in comparison to their growth as monoculture (FIG. 27). Number of biofilm-embedded bacteria were between 10⁶-10^{7 CFU/mL.}

Findings from the LRh-LG co-culture experiment were latter verified by repeating the experiment in a medium scale set-up. These set of experiments demonstrated that co-culture of these two bacterial strains combinations (LJ & LRh or LG & LRh) displayed a clear advantage for reducing number of particles while increasing total number of bacteria in biofilm. This is a major advantage when reducing the size of the biofilm carries, e.g., a suppository or tablet.

Co-Culture with More than Two Bacterial Strains

In this experiment the inventors inoculated the following bacteria combination: LRh-LJ-LG or LRh-LJ-LG-LCr (hereafter ‘combination-A’ and ‘combination-B’ respectively; FIG. 7). When LRh, LJ and LG were culture together, the number of bacteria in biofilm, for each strain, was between 10⁷ to 10⁸ CFU/mL. The bacteria strains were inoculated in FG1 broth, hence the higher number of biofilm cells compared to the former co-culture experiment that was done with MRS. At pH 3.5, all bacteria strains exhibited high tolerance, regardless the culturing time. However, resistance of LJ bacterial population to pH 3.5 decreased with incubation time. At pH 2, while LG survival was not significantly different from its survival at pH 3.5, LJ and LRh displayed poor survival rate. These results were in accordance with findings from former experiments when bacteria were grown alone. These results indicate that these strains did not inhibit or suppress each other biofilm populations.

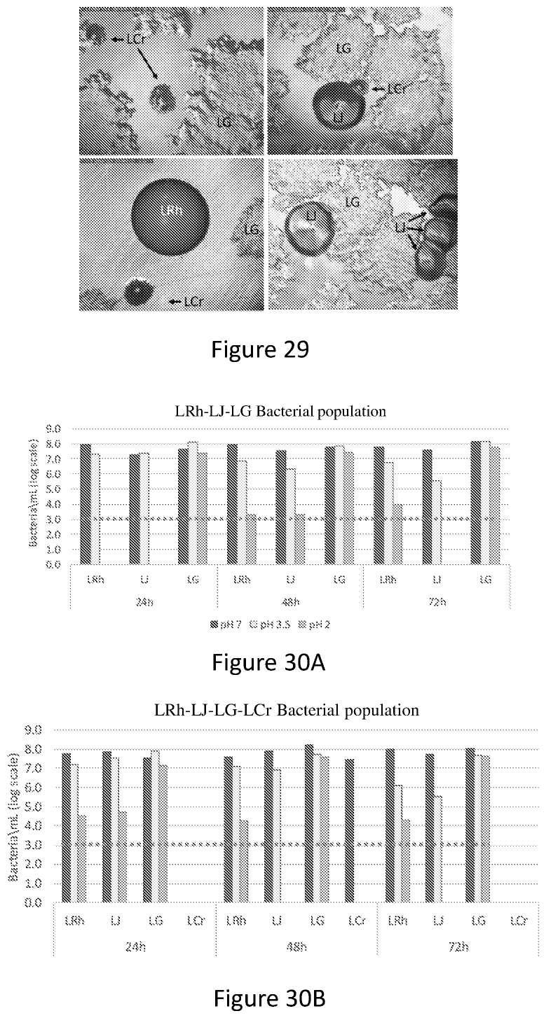

LRh, LJ and LG bacterial population, from the second co-culture mixture, combination-B, showed no difference in growth and resistance to pH 3.5 compare to combination-A. However, LJ and LRh appeared to better withstand pH 2, specifically after 24 hours of incubation. LCr bacterial population, on the other hand, appeared on the MRS agar plates only at the second day of incubation. The high viability of LCr bacterial population was similar to LJ bacterial population (10⁷ CFU/mL). Likely, growth of LCr on the MRS agar plates were suppressed by the growth of the other bacteria strains. (FIGS. 29-30).

Next, bacterial population from combination-B were plated on Bifidobacterium agar plates as well.

Taken together, in both co-culture combinations, the best result as regard to the bacterial population growth and pH resistance was obtained after 24 hours of incubation. However, the relatively high survival, at the low pH treatments, of LRh, LG and LJ from combination-B, suggests this specific combination of bacteria strains, which included LCr, contributed to biofilm development of these bacterial populations. (FIGS. 29-30).

### Order of Co-Culturing

To test the order of bacteria addition to the mixture of matrix:media, at the beginning of the incubation, the inventors compare the addition of the weaker/slow-growing strains and then the stronger/faster-growing strains with the opposite combination (first the stronger and then the weaker). This shows if there is a benefit in the order of the strains added to the fermentation on the formation of biofilm by weaker strains.

Bacterial culture of LRh, LCr, LJ and LG, in this experiment is prepared similarly to the described in the Material and Method section, with the following adjustment: when planktonic bacteria from each strain is added to the mixture of growth medium and matrix, at the beginning of the experiment, each strain is added one after the other with a gap of 0.5-1 hour between them. There are two treatments:

1) Addition of the anaerobic/slow-growing strains first and then the aerobic/faster-growing strains, LCr→LJ→LG→LRh;
2) Addition of the stronger/faster-growing strains first and then slow weaker/slow-growing the strains, LRh→LG→LJ→LCr.

Analysis of bacterial population growth is carried out every 24 hours incubation during a total of 72 hours.

Bacteria strains that is grown in co-culture is compared to monoculture growth and differences in metabolites are analyzed using metabolomics techniques.

Bacterial population culture of LRh, LCr, LJ and LG, grown either together as coculture or each strain separately, are prepared as described in the Material and Method section. At the end of 72 hours of incubation, the growth medium where the bacterial population are cultured, are centrifuge at maximum speed, for 10 min to remove debris and planktonic bacteria. The supernatant is transferred to a clean tube and are centrifuge again (same conditions). The supernatant is kept at −80° C. till analysis of metabolism. Additionally, the effect of addition of different supplements (for example carbohydrates such as fructooligosaccharide and trace elements such as iron) to the growth media on coculture growth compare to monoculture is investigated.

### EXAMPLE 6

### Particle Sizes and Bacterial Affinity to the Particles

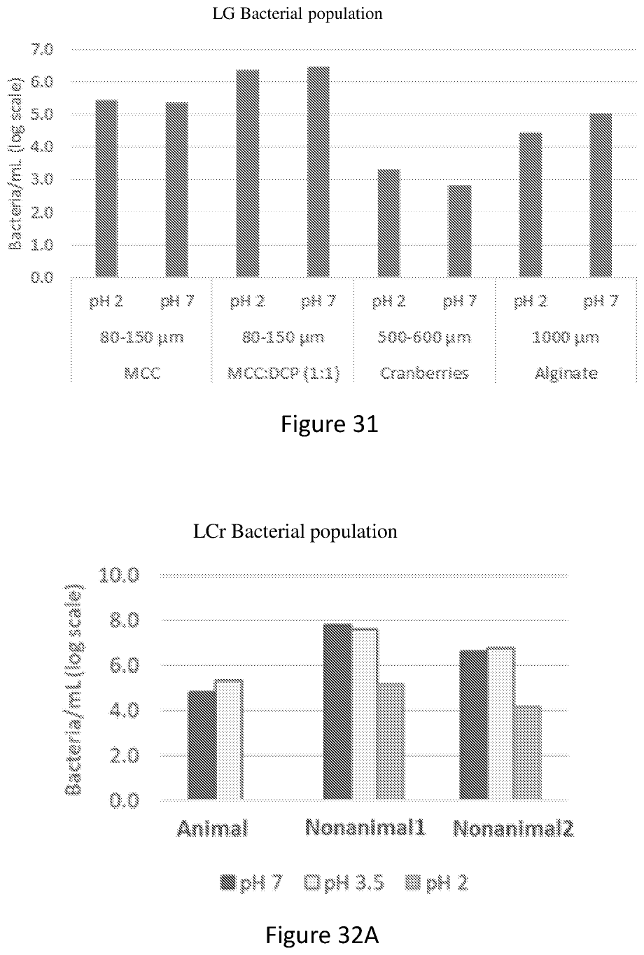

After 24 hours of incubation of LG planktonic cells with different particles, LG bacterial population growth and development was as follows Microcrystalline cellulose: Di calcium phosphate (MCC:DCP)>MCC>Alginate>Cranberries. Based on these results the inventors suggest that the combination of both reduced particle size and the type of matrix positively influenced LG Bacteria in form of biofilm growth and development: 1) particle sizes—smaller-particles size (higher surface to volume ratio) may allow more bacteria to attach per particle volume, thus improving bacteria population growth: bacterial population was enhanced on MCC or MCC:DCP combination (80-150 μm) compare to alginate beads (1000 μm); 2) type of matrix—the inventors suggest here two possible explanations, without wishing to be bound to any particular theory, for the contribution of the specific matrix to biofilm growth and formation. (FIG. 31).

Despite alginate being twice as big as compared to cranberries, LG bacteria population growth was higher when inoculated with alginate. Another example is the difference in the number of LG biofilms cells between MCC to MCC:DCP. Few studies have shown that exogenous Calcium ions can promote biofilm formation, hence the presence of soluble DCP particles (dicalcium phosphate) and subsequently calcium ions may have enhanced the growth and development of LG biofilm.

### EXAMPLE 7

### The Effect of Animal and Non-Animal Medium on Bacterial Population Growth

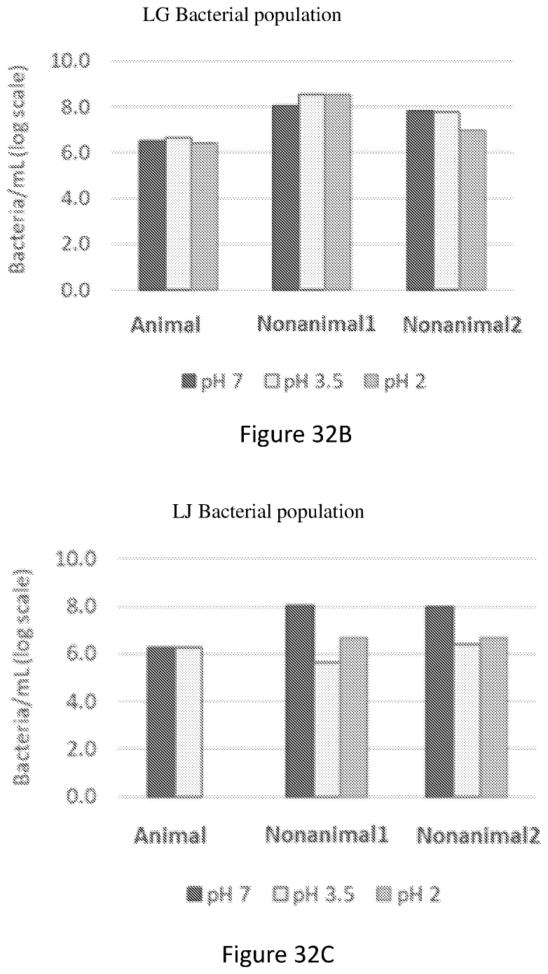

The effect of different animal-based and non-animal-based growth mediums on bacterial population growth and developmental state of LG, LRh, LCr and LJ was assessed (FIGS. 32A-32C). Lactobacillus sp. were grown on either animal-based broth (hereafter Animal) and/or nonanimal-based broth (hereafter Non-animal1 and Non-animal2).

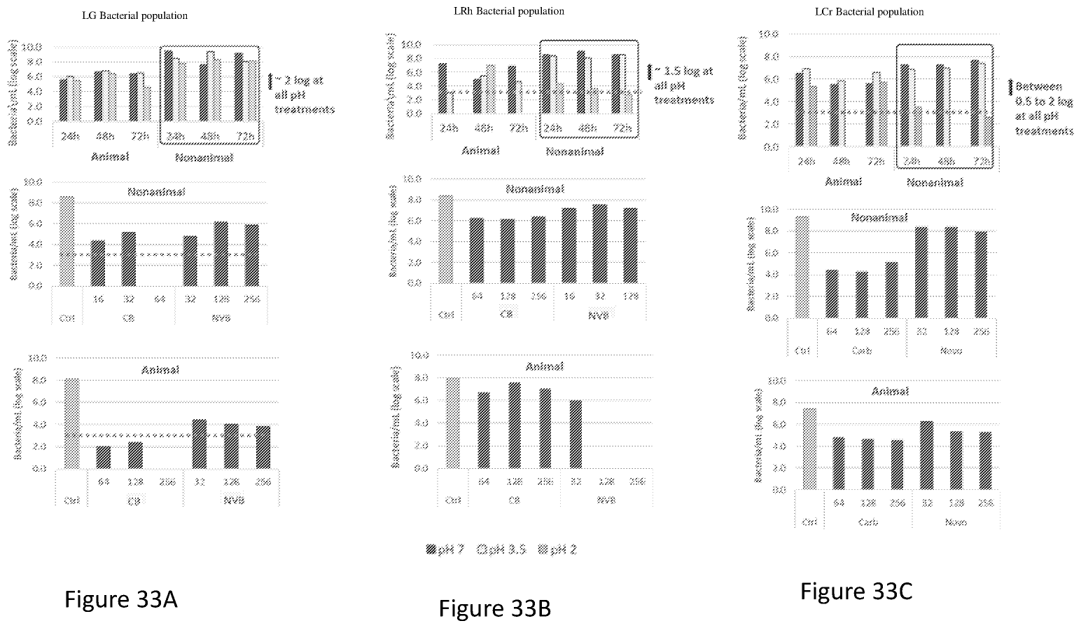

In the first experiment, after 24 hours of incubation in the different growth media, the bacterial population growth of LG and LCr and their resistance to pH were as follows Non-animal1>Non-animal2>Animal whereas LJ displayed a growth and resistance pattern where Non-animal1=Non-animal2>Animal (FIGS. 33A-33C). Based on this experiment, FG1 broth was the medium of choice to be tested in medium-scale set-up or with different strains.

The next set of experiments was used to evaluate the effect of Non-animal1 broth on growth and biofilm development of LRh, LG and LCr, in comparison to Animal broth. Here, both resistance assays, pH and antibiotics were tested, and these experiments were carried out using one of the two experimental designs (small-scale or medium-scale set-up; FIG. 12A-C) Overall, number of viable biofilm cells was more pronounced when grown in Non-animal1 broth; an increase of ˜1-2 log was recorded for all tested bacteria strains. Nonetheless, while survival and/or growth of LG in the presence of both acidic pH and antibiotics was substantially better when cultured in Non-animal1 compared to Animal. Number of LCr- and LRh-embedded biofilm cells after exposure to pH 3.5 and NVB antibiotic, but not pH 2 and CB antibiotic, was higher in LCr that was inoculated in Nonanimal1-based medium compared to inoculation in Animal-based medium. This result was correlated with increasing amount of biofilm cells (pH 7). Taken together, growth with Nonanimal-based medium showed a clear advantage over Animal-based medium in the number of bacteria in biofilm as well as biofilm development. This was recorded for all vaginal bacteria tested in this application. (FIG. 33).

### EXAMPLE 8

### Bifidobacterium adolescentis Mono- vs Co-Culture

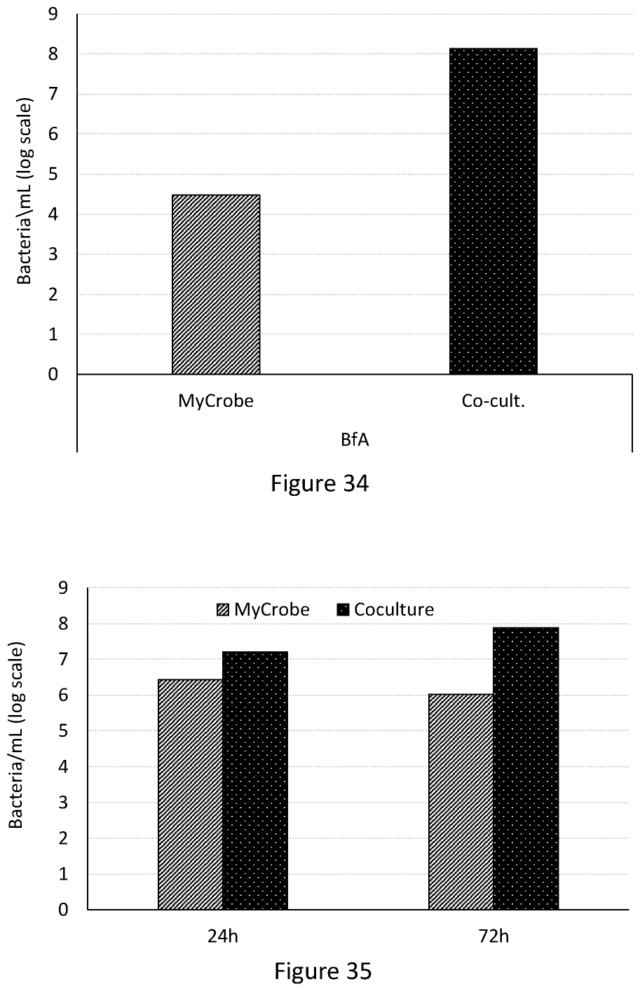

The inventors examined the growth performance of B. adolescentis (BfA) in a monoculture compared to its biofilm coculture growth with B. breve and B. longum sub.longum. BfA showed an improved growth after 72 h when cocultured with BLL and BfBr compared to its growth alone (FIGS. 34). BfA showed an improved growth after 24 h and 72 h when cocultured with BfBr compared to its growth alone (FIGS. 35).

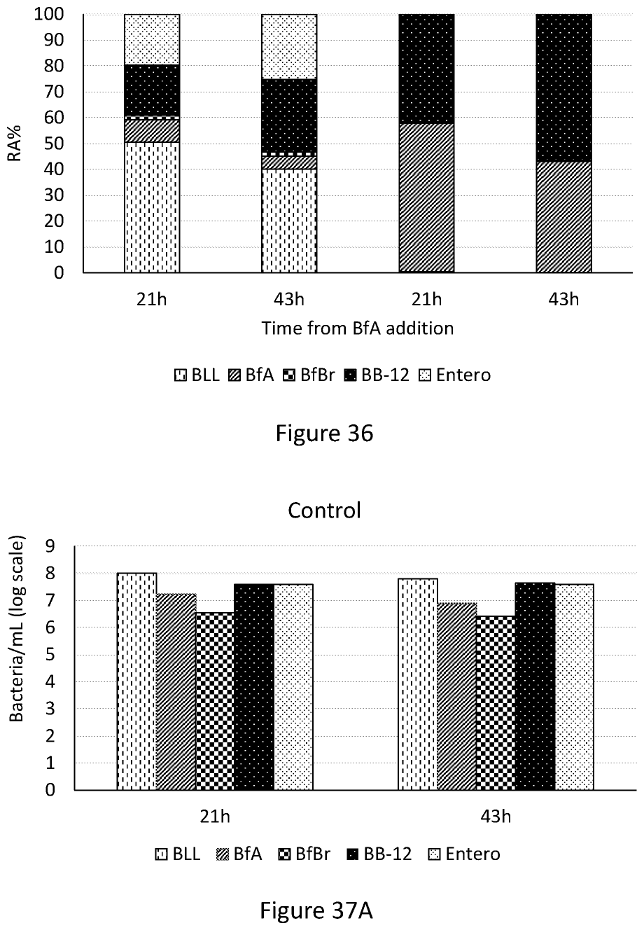

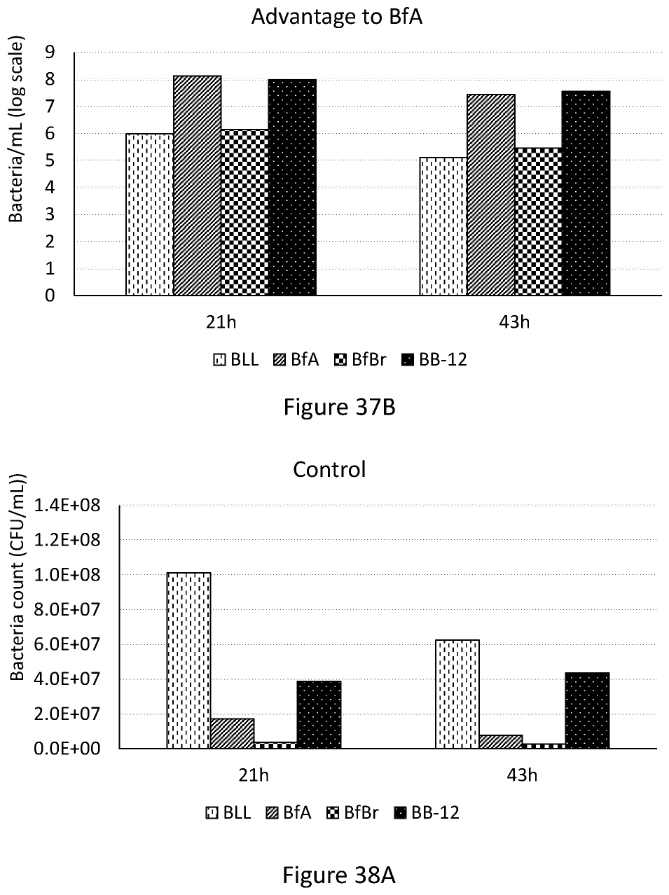

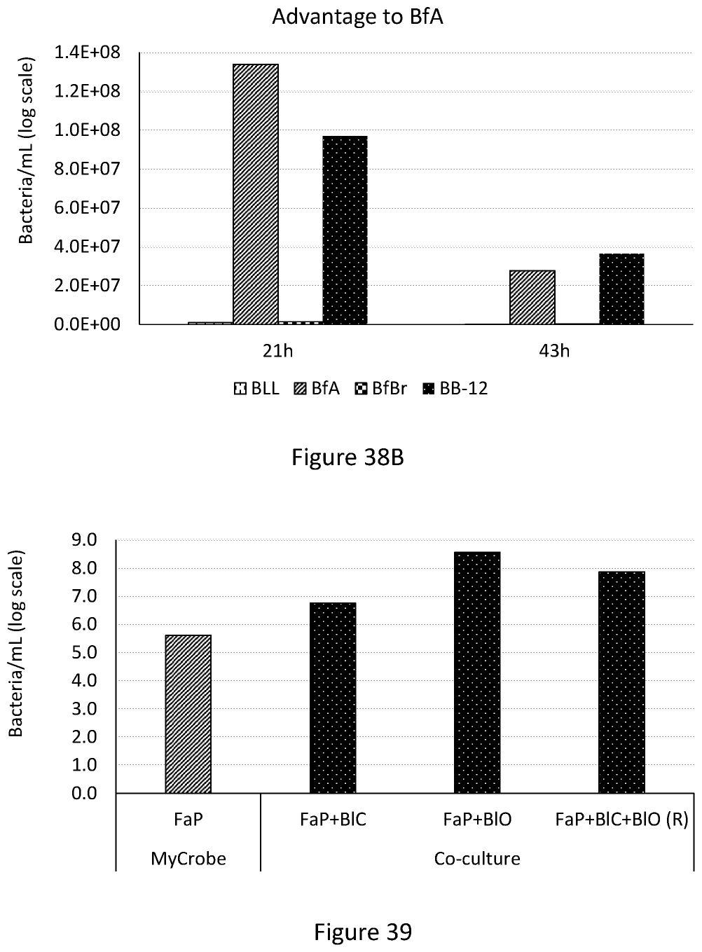

The inventors further examined whether time of addition may improve B. adolescentis growth in coculture. BfA was added either in parallel with BLL and BfBr (‘control’) or 24 h before the addition of BLL and BfBr (‘Advantage to BfA’). Higher relative abundance of BfA was observed when it was added 24 h prior to the addition of BLL, BB-12 and BfBr (FIG. 36). Improved growth of BfA (˜1 log difference) was observed when it was added 24 h prior to the addition of BLL, BB-12 and BfBr (Advantage to BfA) compare to its addition in parallel to the other Bifidobacterium species (Control; FIGS. 37-38).

### EXAMPLE 9

### Faecalibatcerim Prausnitzii Mono- vs. Co-Culture

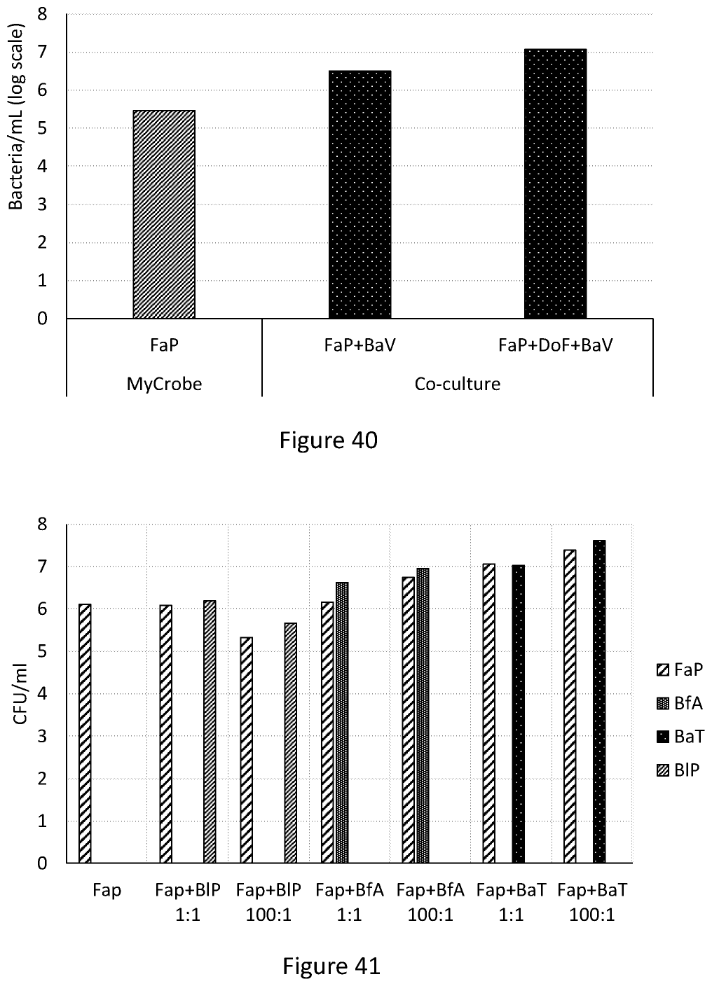

The inventors examined the growth performance of F. prausnitzii (FaP) in a monoculture compared to its biofilm coculture growth with Blautia obeum (BlO), Blautia coccoides (BlC), or both. FaP showed an improved growth when cocultured with either BlO, BlC or both for 48 h, compare to its growth alone (FIG. 39). FaP also showed an improved growth when cocultured with either Bacteroides vulgatus (BaV), or BaV and Dorea (Eubacterium) formicigenerans (DoF), compare to its growth alone (FIG. 40).

The inventors further examined FaP growth compared to its growth with either BlP, BfA or Bacteroides thetaiotaomicron (BaT). Also, the inventors examined the effect of higher initial OD of FaP on its growth. FaP was found to have an improved growth when coculture with either BfA or BaT for 48 h (FIG. 41).

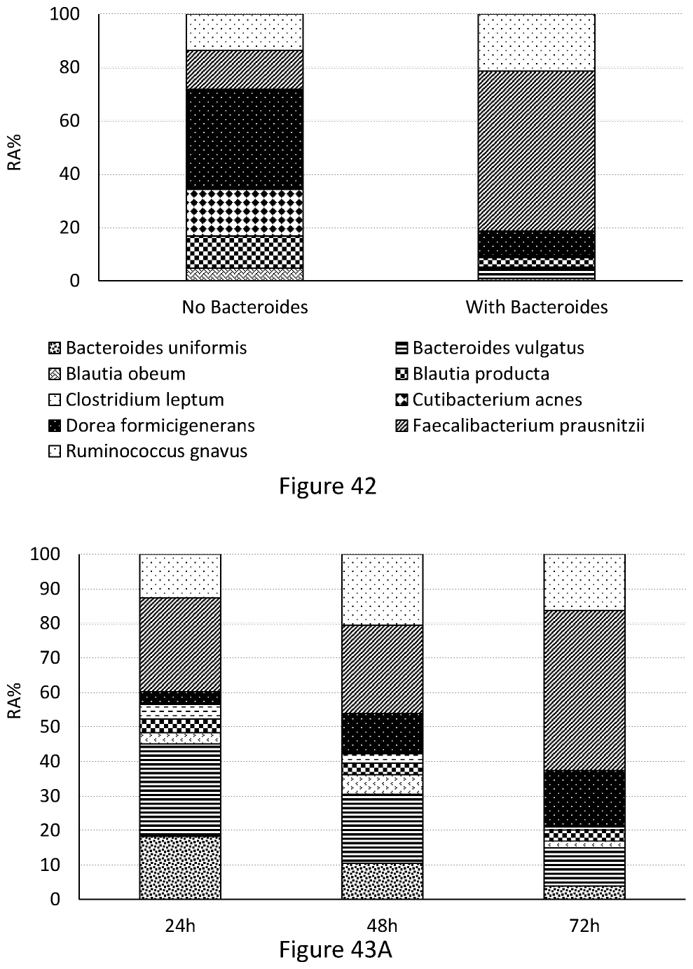

The inventors further examined FaP relative abundance during 72 h of incubation when coculture with few species from the Clostridiales and Bacteroides families. When FaP was cocultured with Bacteroides spp. a clear advantage was observed compared to its growth in coculture with only Clostridiales spp. (FIG. 42).

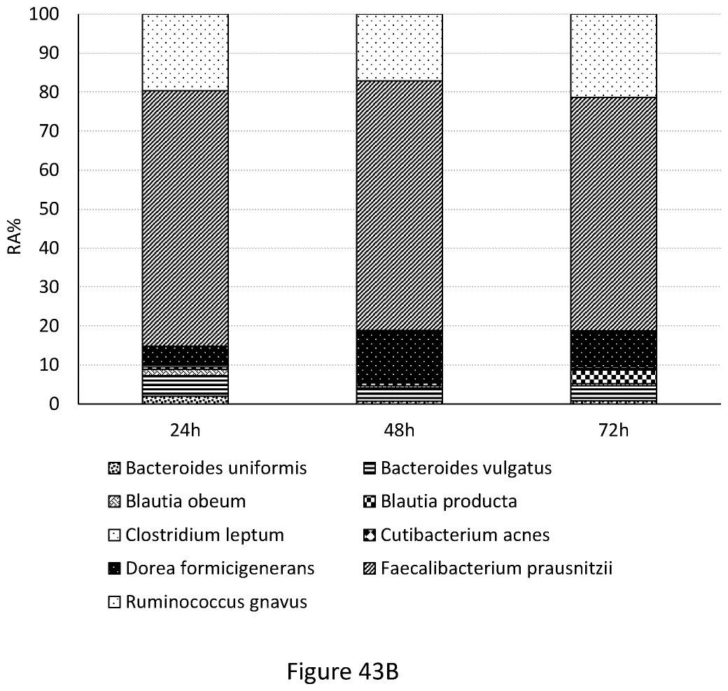

Further, the inventors examined FaP relative abundance in planktonic and biofilm phase during 72 h of incubation when coculture with few species from the Clostridiales and Bacteroides families. FaP was found to have a higher relative abundance in biofilm (FIG. 43B) compared to the planktonic form (FIG. 43A) during 72 h of growth.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A composition comprising a first lipophilic carrier and a co-cultured probiotic bacteria in the form of a dried biofilm comprising Lactobacillus crispatus and at least one additional bacterial species selected from the group consisting of: L. gasseri, L. jensenii, and L. rhamnosus, and a first agent comprising: an antibiotic agent, a pH adjusting agent, or both.

2. The composition of claim 1, wherein said co-cultured probiotic bacteria in the form of a dried biofilm and said first agent comprising an antibiotic agent are homogeneously dispersed within said composition.

3. The composition of claim 1, wherein said co-cultured probiotic bacteria in the form of dried biofilm is 10% to 50% (w/w) of the total composition.

4. The composition of claim 1, wherein said co-cultured probiotic bacteria in the form of dried biofilm is attached to a particle.

5. The composition of claim 4, wherein said particle is selected from the group consisting of: seeds, MCC, dicalcium phosphate, a polysaccharide, and any combination thereof.

6. The composition of claim 1, further comprising a second layer.

7. The composition of claim 6, wherein said second layer comprises a second lipophilic carrier, a second agent or both.

8. The composition of claim 7, wherein the release of said co-cultured probiotic bacteria in the form of dried biofilm is slower than the release of said second agent.

9. The composition of claim 7, wherein any one of said first agent and said second agent is an antibiotic.

10. The composition of claim 1, further comprising a stabilizer, a preservative, a lubricant, a viscosity modifying agent, a buffering agent, fatty acids, and combinations thereof.

11. The composition of claim 1, for use in the treatment of bacterial vaginosis.

12. A composition comprising:
a co-cultured probiotic bacteria in the form of a dried biofilm comprising:
a. Faecalibacterium prausnitzii and at least one additional bacterial species selected from the group consisting of: Blautia obeum, Bl. coccoides, Bacteroides vulgatus, and Dorea (Eubacterium) formicigenerans;
b. Lactobacillus crispatus and at least one additional bacterial species selected from the group consisting of: L. gasseri, L. jensenii, and L. rhamnosus; or
c. Bifidobacterium adolescentis and at least one additional bacterial species selected from the group consisting of: Bif. longum sub.longum, and Bif. Breve.

13. The composition of claim 1, formulated for a delivery route selected from the group consisting of: oral, vaginal, rectal, and topical.

14. The composition of claim 1, being in the form of a suppository.

15. A method for modulating the flora in a subject in need thereof, comprising administering to said subject a therapeutically effective amount of the composition of claim 1, thereby modulating the flora in the subject.

16. The method of claim 15, wherein said flora is a vaginal flora, a gut flora, or a skin flora.

17. The method of claim 15, wherein said modulating is restoring the native flora of said subject.

18. The method of claim 15, for preventing or treating a dysbiosis related condition or an intestinal and metabolic disease in a subject in need thereof.

19. The method of claim 18, wherein said dysbiosis related condition or an intestinal and metabolic disease is selected from the group consisting of: bacterial vaginosis, a urogenital infection, ulcerative colitis, inflammatory bowel disease (IBD), Crohn's disease, colorectal cancer, obesity, and celiac disease.

20. A method for preparing the composition of claim 1 comprising the steps of:
a. inoculating a growth medium comprising a particle with a L. crispatus;
b. incubating the particle with L. crispatus from step (a) under conditions suitable for allowing L. crispatus to attach to said particle;
c. inoculating the particle of step (b) with at least one additional bacterial species; and
d. culturing the inoculated particle of step (c) under conditions suitable for forming a biofilm comprising: L. crispatus and at least one additional bacterial species.

21.-26. (canceled)
